(19) United States
(12) Patent Application Publication
(10) Pub. No.: US 2025/0000788 A1
(43) Pub. Date: Jan. 2, 2025

(54) BIOCOMPATIBLE DEVICES FOR CELL-BASED THERAPIES AND RELATED METHODS

(71) Applicant: SIGILON THERAPEUTICS, INC.

(72) Inventors: Sofia Brites Boss, Boston, MA (US); Christopher P. Hencken, Boston, MA (US); Hozefa Bandukwala, Southborough, MA (US); Robert James Miller, East Bridgewater, MA (US); Omid Veiseh, Bellaire, TX (US); Devyn McKinley Smith, Barrington, RI (US)

(21) Appl. No.: 18/274,134

(22) PCT Filed: Jan. 26, 2022
(86) PCT No.: PCT/US2022/013945

§ 371 Date: Jul. 25, 2023

Related U.S. Application Data
(60) Provisional application No. 63/141,905, filed on Jan. 26, 2021.

(51) Int. Cl. A61K 9/00; A61K 9/50; A61K 31/5685; A61K 31/573; A61K 31/58; A61K 35/545; A61K 38/13; A61K 38/17; A61K 38/20; A61K 47/61; A61K 47/69

(52) U.S. Cl. CPC A61K 9/0024; A61K 9/5084; A61K 31/5685; A61K 31/573; A61K 31/58; A61K 35/545; A61K 38/13; A61K 38/1709; A61K 38/2066; A61K 47/61; A61K 47/6925

(57) ABSTRACT

Described herein are implantable devices comprising living cells and an extended release formulation of a glucocorticoid.

### BACKGROUND

Treating chronic and genetic diseases by implanting living cells that produce a therapeutic substance capable of treating such diseases has exciting potential to improve the health of patients with such diseases. To fully achieve this potential, the implanted cells must be protected from the patient's immune response, so that they remain viable, and the implanted cells must also be capable of producing therapeutic levels of the desired therapeutic substance for several weeks, months or even longer. One exploratory approach for delivering such cell-based therapies is to encapsulate the therapeutic-producing cells into semi-permeable devices (e.g., hydrogel capsules), with the objective that the device structure isolates the therapeutic-producing cells from immune system cells, while allowing entry of nutrients for the therapeutic-producing cells and exit of the therapeutic from the device. However, most biomaterials typically used in encapsulated cell therapy (ECT) devices elicit a foreign body response (FBR) to the implanted device, which typically results in pericapsular fibrotic overgrowth (PFO) on the device, which in turn can result in reduced amounts of therapeutic substances exiting the ECT device and death of the encapsulated cells. A need exists to improve upon this general approach to achieve increased implant biocompatibility.

### SUMMARY

The present disclosure provides ECT devices that continuously release a glucocorticoid compound (e.g., as defined herein) in an amount and for a sufficient time period after implant to mitigate the FBR, thereby reducing formation of PFO on devices containing cells that are allogeneic or xenogeneic to the recipient.

Described herein is an implantable device comprising at least one cell-containing compartment comprising a living cell or a plurality of living cells and an extended release formulation of a glucocorticoid compound. The extended release formulation and device are configured to provide continuous, local release of the glucocorticoid compound from the device during a release period (e.g., at least any of thirty, sixty, ninety or 120 days) after the device is implanted into an immune-competent subject. In an embodiment, the glucocorticoid compound is released in an amount effective to inhibit PFO formation on the device during the entire release period. In an embodiment, the quantity of PFO on the implanted device at the end of the release period is at least 10%, 20%, or 30% lower than the quantity of PFO on an implanted control device at the end of the release period. In an embodiment, the control device lacks the extended release formulation, but is otherwise identical in composition and structure to the device comprising the extended release formulation.

In an embodiment, the extended release formulation comprises a plurality of particles of the glucocorticoid compound suspended in a hydrogel. In an embodiment, the particles comprise the glucocorticoid compound in crystalline form.

In an embodiment, the glucocorticoid compound is a compound of Formula IV:

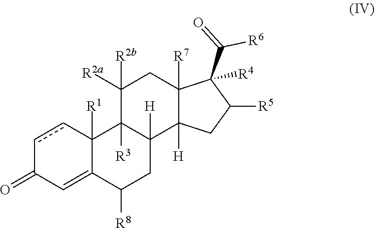

or a pharmaceutically acceptable salt, ester, hydrate, tautomer, or prodrug thereof, wherein variables R¹, R^{2a}, R^{2b}, R³, R⁴, R⁵, R⁶, R⁷, and R⁸, as well as related subvariables, are defined herein. In an embodiment, the compound of formula (IV) is triamcinolone hexacetonide (TAH), fluticasone furoate (FF), fluticasone propionate (FP) or mometasone furoate (MF). In an embodiment, the glucocorticoid compound is not dexamethasone, prednisone, methylprednisolone, prednisolone, hydrocortisone, or fludrocortisone. In an embodiment, the glucocorticoid compound is TAH. In an embodiment, the glucocorticoid compound is FF or MF. In an embodiment, the glucocorticoid compound is FF.

In an embodiment, FBR mitigation is enhanced by including an afibrotic compound (e.g., as defined herein) on the exterior surface of the device. In an embodiment, the quantity of PFO on a device comprising both FBR mitigating substances (the extended release formulation of the glucocorticoid compound and the afibrotic compound) at 30 days after implant in an immune-competent subject is at least 25%, 50%, 75%, 80% or 90% lower than the quantity of PFO on an implanted control device at 30 after implant in an immune-competent subject. In an embodiment, the control device lacks the extended release formulation, but is otherwise identical in composition and structure to the device comprising the extended release formulation, i.e., the control device includes the afibrotic compound on its surface.

In an embodiment, the afibrotic compound is a compound of Formula (I):

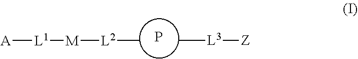

or a pharmaceutically acceptable salt thereof, wherein the variables A, L¹, M, L², P, L³, and Z, as well as related subvariables, are defined herein. In an embodiment, the quantity of PFO on an implanted device comprising both FBR mitigating substances (the extended release formulation of the glucocorticoid and the afibrotic compound) is at least 25%, 50%, 75%, 80% or 90% lower than the quantity of PFO on an implanted control device at the end of the release period. In an embodiment, the control device lacks the extended release formulation, but is otherwise identical in composition and structure to the device comprising the extended release formulation.

In an embodiment, the device is configured as a two-compartment hydrogel capsule (e.g., a hydrogel sphere) in which an inner compartment comprising the cells is completely surrounded by a barrier compartment and one or both of the compartments comprise an extended release formulation of the glucocorticoid compound. In an embodiment, the barrier compartment is substantially free of the extended release formulation. In some embodiments, the barrier compartment comprises a polymer covalently modified with an afibrotic compound, e.g., a compound of Formula (I).

In an embodiment, the cells in the device are mammalian cells genetically modified to express and secrete one or more therapeutic substances during at least part of the release period. In an embodiment, the cells express and secrete the therapeutic substance(s) during substantially all of the release period and for at least 30 days, 60 days, 120 days or longer after the end of the release period.

In an embodiment, the cells in the device are allogeneic to an intended recipient of the device (e.g., cells derived from human cells and a human recipient). In an embodiment, the cells in the device are xenogeneic to the intended recipient of the device (e.g., cells derived from human cells and a non-human mammalian recipient or cells derived from non-human mammalian cells and a human recipient).

In an embodiment, a device described herein, or a plurality of the device, is combined with a pharmaceutically acceptable excipient to prepare a device preparation or a composition which may be administered to a subject (e.g., into the intraperitoneal cavity) in need of treatment with the therapeutic substance(s) produced by the device.

In another aspect, the present disclosure features a method of providing a substance (e.g., a therapeutic agent) to a subject, comprising administering to the subject a device comprising (ii) an extended release formulation of a glucocorticoid compound, as described herein, and (ii) a cell capable of producing the substance (e.g., therapeutic agent). In some embodiments, the substance is a therapeutic agent, e.g., a protein (e.g., a blood clotting factor (e.g., a Factor VIII protein), an enzyme (e.g., alpha-L-iduronidase (IDUA), or a hormone (e.g., insulin)).

In another aspect, the present disclosure features a method of treating a disease, disorder, or condition in a subject with a therapeutic agent that is capable of treating the disease, disorder or condition, the method comprising administering to the subject a device comprising (i) an extended release formulation of a glucocorticoid compound, as described herein, and (ii) a cell capable of producing the therapeutic agent. In some embodiments, the disorder is a blood clotting disorder (e.g., Hemophilia A), a lysosomal storage disorder (e.g., Fabry Disease, MPS-1), an endocrine disorder, diabetes, or a neurodegenerative disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows bright filed micrographs of two-compartment alginate hydrogel capsules which include an alginate modified with an afibrotic compound (“afibrotic alginate”) in the outer compartment and contain cyclosporine A particles in the inner compartment.

FIG. 1B illustrates the kinetics of cyclosporine release from capsules with the same configuration as the FIG. 1A capsules incubated in a normosol solution at 37 degrees Celsius.

FIG. 2A shows bright filed micrographs of two-compartment hydrogel capsules which include the afibrotic alginate in the outer compartment and contain TAH particles in the inner compartment.

FIG. 2B illustrates the kinetics of TAH release from capsules with the same configuration as the FIG. 2A capsules incubated in a normosol solution at 37 degrees Celsius.

FIG. 3A shows fluorescent micrographs of two-compartment hydrogel capsules which include the afibrotic alginate in the outer compartment and contain human cells genetically modified to express and secrete human alpha-galactosidase (GLA) in the inner compartment either without (Control) or with one of several immunosuppressive drugs, with the viability of the cells indicated by Calcein-AM (Green) staining.

FIG. 3B illustrates GLA production by hydrogel capsules with the same configuration as the FIG. 3A capsules during 18 days of culture in vitro.

FIG. 4A shows brightfield micrographs showing extensive PFO on hydrogel capsules with the same configuration as in FIG. 3A that were retrieved 20 days after implantation into the peritoneum of immune-competent C57Bl/6 mice.

FIG. 4B shows computer aided opacity scores illustrating the amount of PFO on the retrieved capsules described in FIG. 4A.

FIGS. 5A-5B show brightfield micrographs of alginate hydrogel capsules which include the afibrotic alginate in the outer compartment and contain the GLA-secreting human cells in the inner compartment without TAH particles (FIG. 5A) or with TAH particles (FIG. 5B) that were retrieved 20 days after peritoneal implantation into immune-competent C57Bl/6 mice.

FIGS. 5C-5L show brightfield micrographs of alginate hydrogel capsules which include the afibrotic alginate in the outer compartment and contain murine IL-10 secreting human cells in the inner compartment without glucocorticoid (left panel) or with particles made from one of four different glucocorticoid compounds (right four panels) that were retrieved 28 days after peritoneal implantation into immune-competent C57Bl/6 mice.

FIG. 6 illustrates hGLA levels at the implantation site (IP lavage) and in plasma of immune-competent C57Bl/6 mice at 20 days after peritoneal implantation of hydrogel capsules with the same configuration as the FIG. 5B capsules.

FIG. 7 illustrates triamcinolone levels at the site of implantation (IP-lavage) and in plasma at 20 days post-implant of hydrogel capsules having the same configuration as in FIG. 5A and FIG. 5B.

FIG. 8A shows the amino acid sequence (SEQ ID NO:1) of an exemplary precursor IL-10 monomer that may be expressed by genetically modified cells described herein, with the signal sequence indicated by underlining.

FIG. 8B shows an exemplary codon-optimized coding sequence (SEQ ID NO:2) for the amino acid sequence in FIG. 8A, with the coding sequence for the signal sequence indicated by shading.

FIG. 8C shows the amino acid sequence (SEQ ID NO:3) of another exemplary precursor IL-10 monomer that may be expressed by genetically modified cells described herein, with the heterologous (HSPG2) signal sequence indicated by underlining.

FIG. 8D and FIG. 8E show exemplary codon-optimized coding sequences (SEQ ID NO:4 and SEQ ID NO:5) for the amino acid sequence in FIG. 8C, with the coding sequence for the signal sequence indicated by shading.

FIG. 8F shows the amino acid sequence (SEQ ID NO:6) of an exemplary variant of precursor IL-10 monomer that may be expressed by genetically modified cells described herein, with the signal sequence indicated by underlining.

FIG. 8G shows an exemplary codon-optimized coding sequence (SEQ ID NO:7) for the amino acid sequence in FIG. 8F, with the coding sequence indicated by shading.

### DETAILED DESCRIPTION

In some embodiments, the present disclosure features an implantable device capable of delivering at least one therapeutic substance to a subject. The therapeutic substance is expressed and secreted by living cells contained in the device. A variety of device configurations and their use for treating a variety of diseases and conditions are contemplated by the present disclosure. Various embodiments will be described below.

### Abbreviations and Definitions

Throughout the detailed description and examples of the disclosure the following abbreviations will be used.

| | |
|---|---|
| CM-Alg | chemically modified alginate |
| CM-LMW-Alg | chemically modified, low molecular weight alginate |
| CM-LMW-Alg-101 | low molecular weight alginate, chemically modified with |
| | Compound 101 shown in Table 2 |
| CM-HMW-Alg | chemically modified, high molecular weight alginate |
| CM-HMW-Alg-101 | high molecular weight alginate, chemically modified with |
| | Compound 101 shown in Table 2 |
| CM-MMW-Alg | chemically modified, medium molecular weight alginate |
| CM-MMW-Alg-101 | medium molecular weight alginate, chemically modified with |
| | Compound 101 shown in Table 2 |
| HMW-Alg | high molecular weight alginate |
| MMW-Alg | medium molecular weight alginate |
| U-Alg | unmodified alginate |
| U-HMW-Alg | unmodified high molecular weight alginate |
| U-LMW-Alg | unmodified low molecular weight alginate |
| U-MMW-Alg | unmodified medium molecular weight alginate |
| 70:30 CM-Alg:U-Alg | 70:30 mixture (V:V) of a chemically modified alginate and |
| | unmodified alginate, e.g., as described in WO2020069429. |

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” means when used herein to modify a numerically defined parameter (e.g., a physical description of a hydrogel capsule such as diameter, sphericity, number of cells encapsulated therein, the number of capsules in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer”, “administering”, or “administration”, as used herein, refer to implanting, absorbing, ingesting, injecting, or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Table 2) is lower than the FBR induced by implantation of an afibrotic-null reference device, i.e., a device that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, TL-6, G-CSF, GM-CSF, TL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to a genetically modified cell or a cell that is not genetically modified. In an embodiment, a cell is an immortalized cell or a genetically modified cell derived from an immortalized cell. In an embodiment, the cell is a live cell, e.g., is viable as measured by any technique described herein or known in the art.

“Conservatively modified variants“or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface Exposure | Asp, Glu, Asn, Aln, His, Arg, Lys |
| | Cys, Ser, Tyr, Pro, Ala, Gly, Trp, Tyr |
| | Met, Ile, Leu, Val, Phe |
| Secondary Structure Propensity | Ala, Glu, Aln, His, Lys, Met, Leu, Arg |
| | Cys, Thr, Ile, Val, Phe, Tyr, Trp |
| | Ser, Gly, Pro, Asp, Asn |
| Evolutionary Conservation | Asp, Glu |
| | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a therapeutic substance that consists essentially of a recited amino acid sequence may also include one or more amino acids, including substitutions in the recited amino acid sequence, of one or more amino acid residues, which do not materially affect the relevant biological activity of the therapeutic substance.

“Derived from”, as used herein with respect to cells, refers to cells obtained from tissue, cell lines, or cells, which optionally are then cultured, passaged, differentiated, induced, etc. to produce the derived cells. For example, mesenchymal stem cells can be derived from mesenchymal tissue and then differentiated into a variety of cell types.

“Device” and “ECD”, as used herein, refer to any implantable object (e.g., a particle, a hydrogel capsule, an implant, a medical device), which contains a cell or cells (e.g., live cells) capable of expressing and secreting a therapeutic substance following implant of the device, and has a configuration that supports the viability of the cells by allowing cell nutrients to enter the device and pore sizes large enough to allow the therapeutic substance to exit the device (e.g., by diffusion).

“Differential volume,” as used herein, refers to a volume of one compartment within a device described herein that excludes the space occupied by another compartment(s). For example, the differential volume of the second (e.g., outer) compartment in a 2-compartment device with inner and outer compartments, refers to a volume within the second compartment that excludes space occupied by the first (inner) compartment.

“Endogenous nucleic acid” as used herein, is a nucleic acid that occurs naturally in a subject cell.

“Endogenous polypeptide,” as used herein, is a polypeptide that occurs naturally in a subject cell.

“Exogenous nucleic acid,” as used herein, is a nucleic acid that does not occur naturally in a subject cell.

“Exogenous polypeptide,” as used herein, is a polypeptide that does not occur naturally in a subject cell, e.g., genetically modified cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Extended-release formulation”, as used herein, means a formulation that releases a glucocorticoid compound from a device in a sustained-release (SR) or controlled-release (CR) profile. SR maintains release of the glucocorticoid compound over a sustained period but not at a constant rate. CR maintains release of the glucocorticoid compound over a sustained period at a nearly constant rate. In some embodiments, extended-release means SR over a period of at least 30 days, at least 45 days, at least any of 50, 60, 70, 80, 90, 100, 110, 120 days or more.

“Genetically-modified cell,” as used herein, is a cell (e.g., an RPE cell) having a non-naturally occurring alteration, and typically comprises a nucleic acid sequence (e.g., an exogenous DNA or RNA) or a polypeptide not present (or present at a different level than) in an otherwise similar cell under similar conditions that is not genetically modified (e.g., lacks the exogenous nucleic acid sequence). In an embodiment, a genetically modified cell comprises an exogenous nucleic acid (e.g., a vector or an altered chromosomal sequence). In an embodiment, a genetically modified cell comprises an exogenous polypeptide. In an embodiment, a genetically modified cell comprises an exogenous nucleic acid sequence, e.g., a sequence, e.g., DNA or RNA, not present in a similar cell that is not genetically modified. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, a genetically modified cell can comprise an exogenous nucleic acid that controls the expression of an endogenous sequence. In an embodiment, a genetically modified cell comprises a polypeptide present at a level or distribution which differs from the level found in a similar cell that has not been genetically modified. In an embodiment, a genetically modified cell comprises an RPE genetically modified to produce an RNA or a polypeptide. For example, a genetically modified cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, a genetically modified cell (e.g., an RPE cell) comprises an exogenous nucleic acid sequence that comprises a chromosomal or extra-chromosomal nucleic acid sequence comprising a sequence that encodes a polypeptide, or which is expressed as a polypeptide. In an embodiment, the polypeptide is encoded by a codon optimized sequence to achieve higher expression of the polypeptide than a naturally-occurring coding sequence. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl_2, pp. W526-W531 (2005)). In an embodiment, a genetically modified cell (e.g., an RPE cell) comprises an exogenous nucleic acid sequence that modulates the conformation or expression of an endogenous sequence. In an embodiment, a genetically modified cell (e.g., RPE cell) is cultured from a population of stably-transfected cells, or from a monoclonal cell line.

“Glucocorticoid”, as used herein, means a naturally occurring or synthetic compound (e.g., hormone, small molecule) which binds to the glucocorticoid receptor expressed by mammalian cells (e.g., human cells), which binding results in up-regulation of the expression of anti-inflammatory proteins (e.g. TGF-beta, interleukin (IL)-1 receptor antagonist, IL-4, IL-10, IL-11, IL-13) and down-regulation of the expression of pro-inflammatory proteins (e.g., interferon gamma, granulocyte-macrophage stimulating factor (GM-CSF), IL-1, IL-12, tumor necrosis factor alpha (TNF-, MCP-1). Non-limiting examples of glucocorticoids include triamcinolone and triamcinolone derivatives (e.g., triamcinolone hexacetonide (TAH), triamcinolone acetonide, triamcinolone benetonide, triamcinolone diacetate), fluticasone and fluticasone derivatives (e.g. fluticasone furoate (FF), fluticasone propionate (FP)), monetasone and mometasone derivatives (e.g., mometasone furoate (MF)), clobetasol and clobetasol derivatives (e.g., clobetasol propionate), beclomethasone and beclomethasone derivatives (e.g., beclomethasone dipropionate), prednisone, prednisolone, methylprednisolone, hydrocortisone, betamethasone, and dexamethasone. In an embodiment, the glucocorticoid is a compound of Formula (IV), as defined herein. In some embodiments, the glucocorticoid is not dexamethasone, prednisone, methylprednisolone, prednisolone, hydrocortisone, or fludrocortisone. In some embodiments, the glucocorticoid is not triamcinolone, triamcinolone acetonide, or clobetasol propionate.

“Peptide”, as used herein, is a polypeptide of less than 50 amino acids, typically, less than 25 amino acids.

“Polymer composition”, as used herein, is a composition (e.g., a solution, mixture) comprising one or more polymers. As a class, “polymers' includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, is a polymer comprising amino acid residues linked through peptide bonds and having at least two, and in some embodiments, at least 10, 50, 75, 100, 150 or 200 amino acid residues.

“Prevention,” “prevent,” and “preventing”, as used herein, refer to a treatment that comprises administering or applying a therapy, e.g., administering a composition of devices encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

“Protein”, as used herein, comprises one or more polypeptide chains of at least 50 amino acids in length. In an embodiment, a protein has two or more polypeptide chains have identical or non-identical amino acid sequences of at least 50 amino acids in length. In an embodiment, the polypeptide chains in a protein are noncovalently associated or covalently joined, e.g., via disulfide bond(s).

“RPE cell”, as used herein, refers to a cell having one or more of the following characteristics: a) it comprises a retinal pigment epithelial cell (RPE) (e.g., cultured using the ARPE-19 cell line (ATCC© CRL-2302™)) or a cell derived therefrom, e.g., by stably transfecting cells cultured from the ARPE-19 cell line with an exogenous sequence that encodes an therapeutic substance or otherwise engineering such cultured ARPE-19 cells to express an exogenous protein or other exogenous substance, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC© CRL-2302Tm)). In an embodiment, an RPE cell described herein is genetically modified, e.g., to have a new property, e.g., the cell is genetically modified to express and secrete one or more therapeutic substances. In other embodiments, an RPE cell is not genetically modified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, means a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a device (e.g., a hydrogel capsule or other particle), means the device has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female), e.g., of any age group, a pediatric subject (e.g., infant, child, adolescent) or adult subject (e.g., young adult, middle-aged adult, or senior adult). In an embodiment, the subject is a non-human animal, for example, a mammal (e.g., a mouse, a dog, a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, a therapy (e.g., a device composition) may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₅), cyclooctenyl (C₅), cubanyl (C₅), bicyclo[1.1.1]pentanyl (C₅), bicyclo[2.2.2]octanyl (C₅), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (I) in a prodrug form. Prodrugs are those compounds that readily undergo chemical changes under physiological conditions to provide the compounds useful for preparing devices in the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R×x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Tautomeric forms may be relevant to the attainment of the optimal chemical reactivity and biologal activity of a compound of interest.

The symbol “ [symbol] ” as used herein refers to a connection to an entity, e.g., a polymer (e.g., hydrogel-forming polymer such as alginate) or surface of an implantable device, e.g., a particle, a hydrogel capsule. The connection represented by “ [symbol] ” may refer to direct attachment to the entity, e.g., a polymer or an implantable element, may refer to linkage to the entity through an attachment group. An “attachment group,” as described herein, refers to a moiety for linkage of a compound of Formula (I) to an entity (e.g., a polymer or an implantable element (e.g., a device) as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alky. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Features of Devices

A device of the present disclosure comprises at least one barrier that prevents immune cells from contacting cells contained inside the device. At least a portion of the barrier needs to be sufficiently porous to allow proteins expressed and secreted by the cells to exit the device. A variety of device configurations known in the art are suitable.

The device (e.g., particle) can have any configuration and shape appropriate for supporting the viability and productivity of the contained cells after implant into the intended target location. As non-limiting examples, device shapes may be cylinders, rectangles, disks, ovoids, stellates, or spherical. The device can be comprised of a mesh-like or nested structure. In some embodiments, a device is capable of preventing materials over a certain size from passing through a pore or opening. In some embodiments, a device (e.g., particle) is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

In an embodiment, the device is a macroencapsulation device. Nonlimiting examples of macrodevices are described in: WO 2019/068059, WO 2019/169089, U.S. Pat. Nos. 9,526,880, 9,724,430 and 8,278,106; European Patent No. EP742818B1, and Sang, S. and Roy, S., Biotechnol. Bioeng. 113(7):1381-1402 (2016).

In an embodiment, the device is a macrodevice having one or more cell-containing compartments. A device with two or more cell-containing compartments may be configured to produce two or more therapeutic substances, e.g., cells expressing a first therapeutic protein would be placed in one compartment and cells expressing a second therapeutic protein would be placed in a separate compartment. WO 2018/232027 describes a device with multiple cell-containing compartments formed in a micro-fabricated body and covered by a porous membrane.

In an embodiment, the device is configured as a thin, flexible strand as described in U.S. Pat. No. 10,493,107. This strand comprises a substrate, an inner polymeric coating surrounding the substrate and an outer hydrogel coating surrounding the inner polymeric coating. The protein-expressing cells are positioned in the outer coating.

In some embodiments, a device (e.g., particle) has a largest linear dimension (LLD), e.g., mean diameter, or size that is at least about 0.5 millimeter (mm), preferably about 1.0 mm, about 1.5 mm or greater. In some embodiments, a device can be as large as 10 mm in diameter or size. For example, a device or particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm.

In some embodiments, a device of the disclosure (e.g., particle, capsule) comprises at least one pore or opening, e.g., to allow for the free flow of materials. In some embodiments, the mean pore size of a device is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of a device is between about 0.1 μm to 10 μm. In some embodiments, the mean pore size of a device is between about 0.1 μm to 5 μm. In some embodiments, the mean pore size of a device is between about 0.1 μm to 1 μm.

In some embodiments, the device comprises a semi-permeable, biocompatible membrane surrounding the genetically modified cells that are encapsulated in a polymer composition (e.g., an alginate hydrogel). The membrane pore size is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

In an embodiment, the device may contain a cell-containing compartment that is surrounded with a barrier compartment formed from a cell-free biocompatible material, such as the core-shell microcapsules described in Ma, M et al., Adv. Healthc Mater., 2(5):667-672 (2012).

Such a barrier compartment could be used with or without the semi-permeable membrane. Cells in the cell-containing compartment(s) of a device of the disclosure may be encapsulated in a polymer composition. The polymer composition may comprise one or more hydrogel-forming polymers. In addition to the polymer composition in the cell-containing compartment(s), the device (e.g., macrodevice, particle, hydrogel capsule) may comprise or be formed from materials such as metals, metallic alloys, ceramics, polymers, fibers, inert materials, and combinations thereof. A device may be completely made up of one type of material, or may comprise other materials within the cell-containing compartment and any other compartments.

In some embodiments, the device comprises a metal or a metallic alloy. In an embodiment, one or more of the compartments in the device (e.g., the first compartment, the second compartment, or all compartments) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing devices, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device comprises a ceramic. In an embodiment, one or more of the compartments in the device (e.g., the first compartment, the second compartment, or all compartments) comprises a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing devices, the amount of ceramic (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment. In such embodiments, the thickness of the second (outer) compartment means the average distance between the outer boundary of the second compartment and the interface between the two compartments, e.g., the average of the distances measured at each of the thinnest and thickest points visually observed in the outer compartment. In some embodiments (e.g., the device is about 1.5 mm in diameter), the thinnest and thickest distances for the outer compartment are between 25 and 110 micrometers (μm) and between 270 and 480 μm, respectively. In some embodiments, the thickness of the outer compartment is greater than about 10 nanometers (nm), preferably 100 nm or greater and can be as large as 1 millimeter (mm). For example, the thickness (e.g., average distance) of the outer compartment in a hydrogel capsule device described herein may be 10 nm to 1 mm, 100 nm to 1 mm, 500 nm to 1 millimeter, 1 micrometer (μm) to 1 mm, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness (e.g., average distance) of the outer compartment is 100 nm to 1 mm, between 1 μm and 1 mm, between 1 μm and 500 μm or between 5 μm and 1 mm. In some embodiments, the thickness (e.g., average distance) of the outer compartment is between about 50 μm and about 100 μm. In some embodiments (e.g., the device is about 1.5 mm in diameter), the thickness of the outer compartment (e.g., average distance) is between about 180 μm and 260 μm or between about 310 μm and 440 μm.

In some embodiments of a two-compartment hydrogel capsule device, the mean pore size of the cell-containing inner compartment and the outer compartment is substantially the same. In some embodiments, the mean pore size of the inner compartment and the second compartment differ by about 1.5%, 2%, 5%, 7.5%, 10%, 15%, 20%, 25%, 30%, 35%, 40%, 45%, 50%, 55%, 60%, 65%, 70%, 75%, or more. In some embodiments, the mean pore size of the device (e.g., mean pore size of the first compartment and/or mean pore size of the second compartment) is dependent on a number of factors, such as the material(s) within each compartment and the presence and density of a compound of Formula (I).

The device may form part of a plurality of substantially the same devices in a preparation (e.g., composition). In some embodiments, the devices (e.g., particles, hydrogel capsules) in the preparation have a mean diameter or size between about 0.5 mm to about 8 mm. In some embodiments, the mean diameter or size of devices in the preparation is between about 0.5 mm to about 4 mm or between about 0.5 mm to about 2 mm. In some embodiments, the devices in the preparation are two-compartment hydrogel capsules and have a mean diameter or size of about 0.7 mm to about 1.3 mm or about 1.2 mm to about 1.8 mm.

In some embodiments, the surface of the device comprises a compound capable of mitigating the FBR, an afibrotic compound as described herein below. For devices comprising a barrier compartment surrounding the cell-containing compartment, the afibrotic compound may covalently modify a polymer disposed throughout the barrier compartment and optionally throughout the cell-containing compartment.

In some embodiments, one or more compartments in a device comprises an afibrotic polymer, e.g., an afibrotic compound of Formula (I) covalently attached to a polymer. In an embodiment, some or all the monomers in the afibrotic polymer are modified with the same compound of Formula (I). In some embodiments, some or all the monomers in the afibrotic polymer are modified with different compounds of Formula (I). In some embodiments in which the device is a 2-compartment hydrogel capsule, the afibrotic polymer is present only in the outer, barrier compartment.

One or more compartments in a device may comprise an unmodified polymer that is the same or different than the polymer in any afibrotic polymer that is present in the device. In an embodiment, the first compartment, second compartment or all compartments in the device comprise the unmodified polymer.

Each of the modified and unmodified polymers in the device may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof. In polymer-containing devices, the amount of a polymer (e.g., by % weight of the device, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, one or more of the modified and unmodified polymers in the device comprises a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer) and polyethylene glycol.

In some embodiments, one or more of the modified and unmodified polymers in the device comprises a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3-Plastics Used in Medical Devices, (2014):21-53.

In some embodiments, one or more of the modified and unmodified polymers in the device comprises a polypropylene. Exemplary polystyrenes include general purpose or crystal (PS or GPPS), high impact (HIPS), and syndiotactic (SPS) polystyrene.

In some embodiments, one or more of the modified and unmodified polymers comprises a comprises a thermoplastic elastomer (TPE). Exemplary TPEs include (i) TPA—polyamide TPE, comprising a block copolymer of alternating hard and soft segments with amide chemical linkages in the hard blocks and ether and/or ester linkages in the soft blocks; (ii) TPC—co-polyester TPE, consisting of a block copolymer of alternating hard segments and soft segments, the chemical linkages in the main chain being ester and/or ether; (iii) TPO—olefinic TPE, consisting of a blend of a polyolefin and a conventional rubber, the rubber phase in the blend having little or no cross-linking; (iv) TPS—styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU—urethane TPE, consisting of a block copolymer of alternating hard and soft segments with urethane chemical linkages in the hard blocks and ether, ester or carbonate linkages or mixtures of them in the soft blocks; (vi) TPV—thermoplastic rubber vulcanizate consisting of a blend of a thermoplastic material and a conventional rubber in which the rubber has been cross-linked by the process of dynamic vulcanization during the blending and mixing step; and (vii) TPZ—unclassified TPE comprising any composition or structure other than those grouped in TPA, TPC, TPO, TPS, TPU, and TPV.

In some embodiments, the unmodified polymer is an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In an embodiment, the unmodified alginate has a molecular weight of 150 kDa-250 kDa and a G:M ratio of ≥1.5.

In some embodiments, the afibrotic polymer comprises an alginate chemically modified with a Compound of Formula (I). The alginate in the afibrotic polymer may be the same or different than any unmodified alginate that is present in the device. In an embodiment, the density of the Compound of Formula (I) in the afibrotic alginate (e.g. amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (I) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described in WO2020069429), and in certain embodiments, the density of a Compound of Formula (I) (e.g., Compound 101) is between about 1.0% w/w and about 3.0% w/w, between about 1.3% w/w and about 2.5% w/w or between about 1.5% w/w and 2.2% w/w.

In alginate-containing devices, the amount of modified and unmodified alginates (e.g., by % weight of the device, actual weight of the alginate) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate.

In an embodiment, the device comprises at least one cell-containing compartment, and in some embodiments contains two, three, four or more cell-containing compartments. In an embodiment, each cell-containing compartment comprises a plurality of cells (e.g., live cells) and the cells in at least one of the compartments are capable of expressing and secreting at least one therapeutic substance (e.g., peptide or protein) when the device is implanted into a subject. In some embodiments, the cells in a single cell-containing compartment express two or more therapeutic substances, e.g., proteins with complementary activities useful for treating a particular disease of interest.

In an embodiment, all the cells in a cell-containing compartment are derived from a single parental cell-type or a mixture of at least two different parental cell types. In an embodiment, all of the cells in a cell-containing compartment are derived from the same parental cell type, but a first plurality of the derived cells are genetically modified to express a first therapeutic substance, and a second plurality of the derived cells are genetically modified to express a second therapeutic substance. In devices with two or more cell-containing compartments, the cells and the therapeutic substance(s) produced thereby may be the same or different in each cell-containing compartment. In some embodiments, all of the cell-containing compartments are surrounded by a single barrier compartment. In some embodiments, the barrier compartment is substantially cell-free.

In an embodiment, cells to be incorporated into a device described herein, e.g., a hydrogel capsule, are prepared in the form of a cell suspension prior to being encapsulated within the device. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In addition to therapeutic substance(s) expressed by the encapsulated cells, a device (e.g., capsule, particle) may comprise one or more exogenous agents that are not expressed by the cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), an active or inactive fragment of a protein or polypeptide, a small molecule, or drug. In an embodiment, the device is configured to release such an exogenous agent.

### Afibrotic (e.g., FBR-Mitigating) Compounds

In some embodiments, the devices described herein comprise at least one compound of Formula (I):

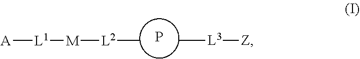

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O), —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1}), cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (I) is a compound of Formula (I-a):

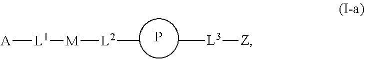

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D}), N(R^{C})C(O)(C₁-C₆-alkylene)-, —N(R^{C})C(O)(C₁-C₆-alkenylene)-, —NCN—, —C(═N(R^{C})(R^{D}))O—, —S—, —S(O), —OS(O)ₓ, —N(R^{C})S(O)ₓ, —S(O)ₓN(R^{C})—, —P(R^{F})_{y}—, —Si(OR^{A})₂—, —Si(R^{G})(OR^{A}y, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²
L² is a bond;
M is absent, alkyl, heteroalkyl, cycloalkyl, heterocyclyl, aryl, or heteroaryl, each of which is optionally substituted by one or more R³
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶ each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (I) and (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) and (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) and (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) and (I-a), M is absent, alkyl, heteroalkyl, aryl, or heteroaryl. In some embodiments, for Formulas (I) and (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂—)₂, wherein z is an integer selected from 1 to 10. In some embodiments, z is an integer selected from 1 to 5. In some embodiments, M is —(OCH₂)₂—, (—OCH₂CH₂—)₂, (—OCH₂CH₂—)₃, (—OCH₂CH₂—)₄, or (—OCH₂CH_{2 5}. In some embodiments, M is —OCH₂CH₂—, (—OCH₂CH₂—)₂, (—OCH₂CH₂—)₃, or (—OCH₂CH₂—)₄. In some embodiments, M is (—OCH₂—)₃. In some embodiments, M is aryl. In some embodiments, M is phenyl. In some embodiments, M is unsubstituted phenyl. In some embodiments, M is

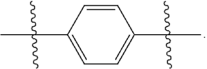 [SMILES: *c1ccc(*)cc1]

In some embodiments, M is

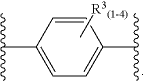

In some embodiments, M is phenyl substituted with 1-4 R³ (e.g., 1 R³). In some embodiments, R³ is CF₃. In some embodiments, for Formulas (I) and (I-a), P is absent, heterocyclyl, or heteroaryl. In some embodiments, for Formulas (I) and (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, or pyrrolyl. In some embodiments, P is imidazolyl. In some embodiments, P is 1,2,3-triazolyl. In some embodiments, P is

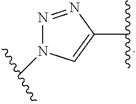 [SMILES: *c1cn(*)nn1]

In some embodiments, P is

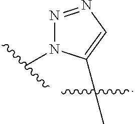 [SMILES: *c1cnnn1*]

In some embodiments, P is

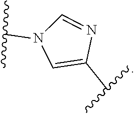 [SMILES: *c1cn(*)cn1]

In some embodiments, P is heterocyclyl. In some embodiments, P is heterocyclyl. In some embodiments, P is a 5-membered heterocyclyl. In some embodiments, P is imidazolidinonyl. In some embodiments, P is

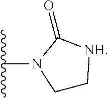 [SMILES: *N1CCNC1=O]

In some embodiments, P is thiomorpholinyl-1,1-dioxidyl. In some embodiments, P is

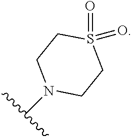 [SMILES: *N1CCS(=O)(=O)CC1]

In some embodiments, for Formulas (I) and (I-a), Z is alkyl, heteroalkyl, cycloalkyl, heterocyclyl, aryl, or heteroaryl. In some embodiments, for Formulas (I) and (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl, 5-membered heterocyclyl, or 6-membered heterocyclyl. In some embodiments, Z is a 6-membered oxygen-containing heterocyclyl. In some embodiments, Z is tetrahydropyranyl. In some embodiments, Z is

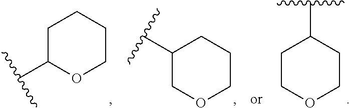

In some embodiments, Z is a 4-membered oxygen-containing heterocyclyl. In some embodiments, Z is

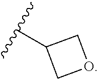 [SMILES: *C1COC1]

In some embodiments, Z is a bicyclic oxygen-containing heterocyclyl. In some embodiments, Z is a bicyclic oxygen-containing heterocyclyl. In some embodiments, Z is phthalic anhydridyl. In some embodiments, Z is a sulfur-containing heterocyclyl

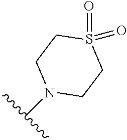 [SMILES: *N1CCS(=O)(=O)CC1]

In some embodiments, Z is a 6-membered sulfur-containing heterocyclyl

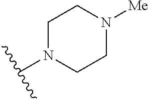 [SMILES: *N1CCN(C)CC1]

In some embodiments, Z is a 6-membered heterocyclyl containing a nitrogen atom and a sulfur atom. In some embodiments, Z is thiomorpholinyl-1,1-dioxidyl. In some embodiments, Z is

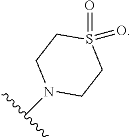 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

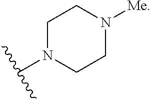 [SMILES: *N1CCN(C)CC1]

In some embodiments, Z is a bicyclic heterocyclyl. In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl In some embodiments, Z is

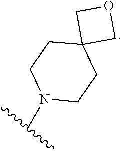 [SMILES: *N1CCC2(CC1)COC2]

In some embodiments, Z is 1-oxa-3,8-diazaspiro[4.5]decan-2-one. In some embodiments, Z is

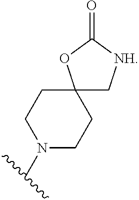 [SMILES: *N1CCC2(CC1)CNC(=O)O2]

In some embodiments, for Formulas (I) and (I-a), Z is aryl. In some embodiments, Z is monocyclic aryl. In some embodiments, Z is phenyl. In some embodiments, Z is monosubstituted phenyl (e.g., with one R⁵). In some embodiments, Z is monosubstituted phenyl, wherein the one R⁵ is a nitrogen-containing group. In some embodiments, Z is monosubstituted phenyl, wherein the one R⁵ is NH₂. In some embodiments, Z is monosubstituted phenyl, wherein the one R⁵ is an oxygen-containing group. In some embodiments, Z is monosubstituted phenyl, wherein the one R⁵ is an oxygen-containing heteroalkyl. In some embodiments, Z is monosubstituted phenyl, wherein the one R⁵ is OCH₃. In some embodiments, Z is monosubstituted phenyl, wherein the one R⁵ is in the ortho position. In some embodiments, Z is monosubstituted phenyl, wherein the one R⁵ is in the meta position. In some embodiments, Z is monosubstituted phenyl, wherein the one R⁵ is in the para position.

In some embodiments, for Formulas (I) and (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) and (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

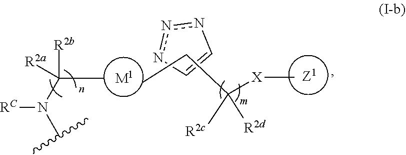

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R^{5′} and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

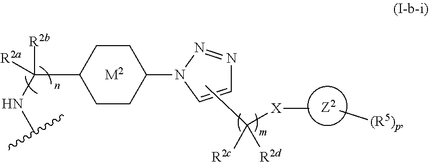

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

[symbol]

In some embodiments, the compound of Formula (I-b-i) is a compound of Formula (I-b-ii):

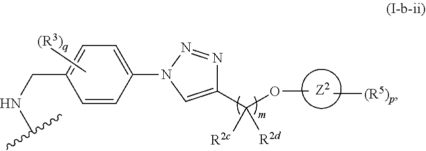

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

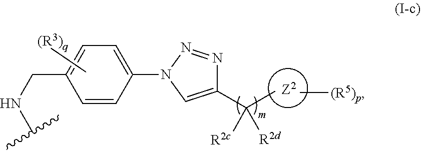

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR, —C(O)OR, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

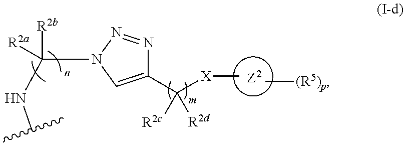

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R [symbol] is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

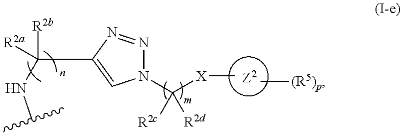

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

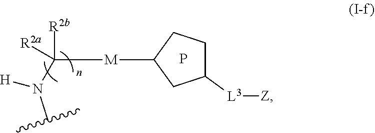

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

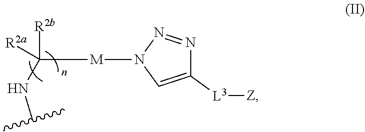

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, [symbol] heteroaryl or —OR, wherein alkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

[symbol]

In some embodiments, the compound of Formula (II) is a compound of Formula (II-a):

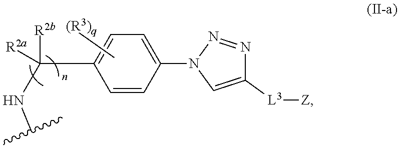

or a pharmaceutically acceptable salt thereof, wherein L³ is alkyl or heteroalkyl, each of which is optionally substituted with one or more R²; Z is hydrogen, alkyl, heteroalkyl, or —OR^{A}, heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1} or —C(O)R^{B1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (III):

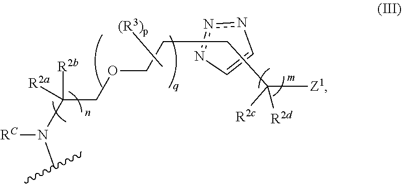

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵ each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl; is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

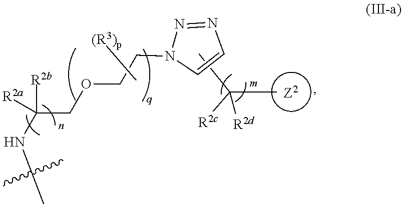

or a pharmaceutically acceptable salt thereof, wherein Ring Z² is cycloalkyl, heterocyclyl, aryl, or heteroaryl, each of which is optionally substituted with 1-5 R⁵ each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and [symbol] are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b).

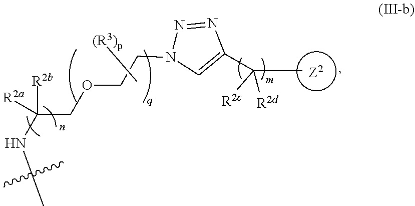

or a pharmaceutically acceptable salt thereof, wherein Ring Z² is cycloalkyl, heterocyclyl, aryl, or heteroaryl, each of which is optionally substituted with 1-5 R⁵ each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n [symbol] 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

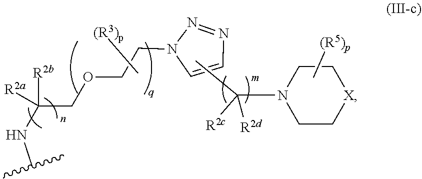

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2′} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4 [symbol] ; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

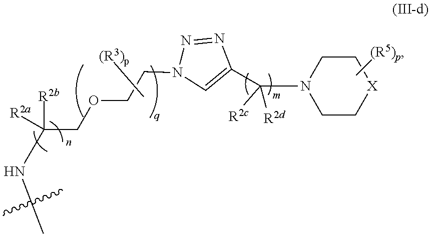

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2′} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4 [symbol] ; q is an integer from 0 to 25; x is 0, 1, or 2; and “ [symbol] ” refers to a connection to an attachment group or a polymer described herein.

In some embodiments, the compound of Formula (I) is a compound of Formula (III-e):

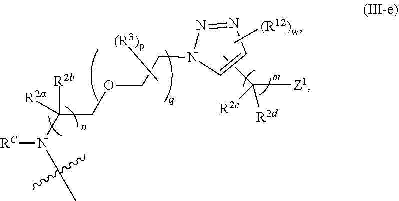

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵ each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; w is 0 or 1; and “ [symbol] ” refers to a connection to an attachment group or a polymer described herein.

In some embodiments, the compound of Formula (I) is a compound of Formula (III-f):

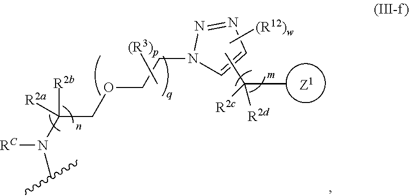

or a pharmaceutically acceptable salt thereof, wherein Ring Z¹ is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; w is 0 or 1; and “ [symbol] ” refers to a connection to an attachment group or a polymer described herein.

In some embodiments, the compound of Formula (I) is a compound of Formula (III-g):

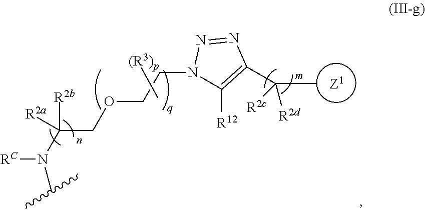

or a pharmaceutically acceptable salt thereof, wherein Ring Z¹ is cycloalkyl, heterocyclyl, aryl, or heteroaryl, each of which is optionally substituted with 1-5 R⁵; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; and “ [symbol] ” refers to a connection to an attachment group or a polymer described herein.

In some embodiments, the compound of Formula (I) is a compound of Formula (III-h):

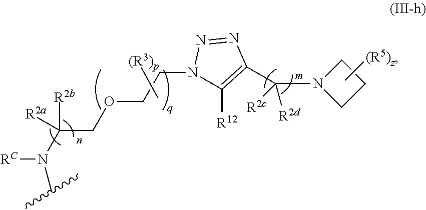

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound of Formula (I) is a compound of Formula (III-i):

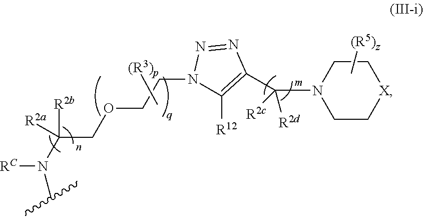

or a pharmaceutically acceptable salt thereof, wherein X is C(R′)(R″), N(R′), or S(O)ₓ; each of R′ and R″ is independently hydrogen, alkyl, or halogen; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹² is halo (e.g., Cl).

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is 0, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl). In some embodiments, the compound of Formula (III-b) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 2, or a pharmaceutically acceptable salt thereof. In some embodiments, the exterior surface and/or one or more compartments within a device described herein comprises a small molecule compound shown in Table 2, or a pharmaceutically acceptable salt thereof.

TABLE 2
Exemplary afibrotic (FBR-mitigating) compounds

| Compound | |
|---|---|
| No. | Structure |
| 100 | 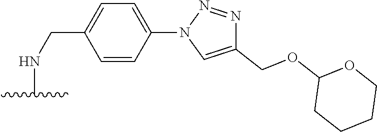 [SMILES: *NCc1ccc(-n2cc(COC3CCCCO3)nn2)cc1] |
| 101 | 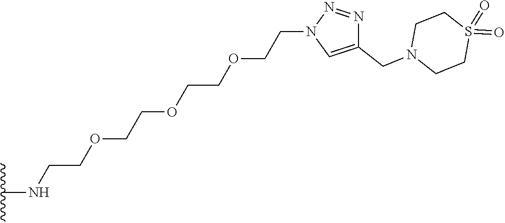 [SMILES: *NCCOCCOCCOCCn1cc(CN2CCS(=O)(=O)CC2)nn1] |
| 102 | 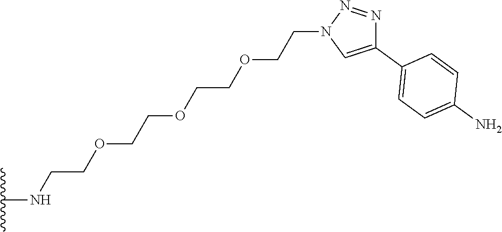 [SMILES: *NCCOCCOCCOCCn1cc(-c2ccc(N)cc2)nn1] |
| 103 | 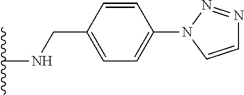 [SMILES: *NCc1ccc(-n2ccnn2)cc1] |
| 104 | 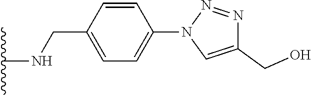 [SMILES: *NCc1ccc(-n2cc(CO)nn2)cc1] |
| 105 | 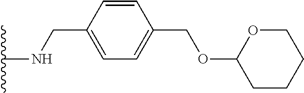 [SMILES: *NCc1ccc(COC2CCCCO2)cc1] |
| 106 | 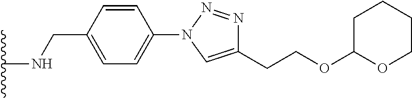 [SMILES: *NCc1ccc(-n2cc(CCOC3CCCCO3)nn2)cc1] |
| 107 | 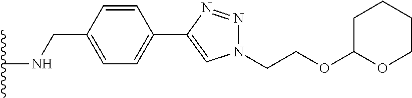 [SMILES: *NCc1ccc(-c2cn(CCOC3CCCCO3)nn2)cc1] |
| 108 | 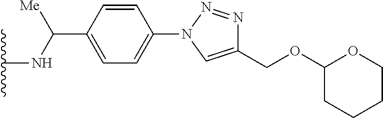 [SMILES: *NC(C)c1ccc(-n2cc(COC3CCCCO3)nn2)cc1] |
| 109 | 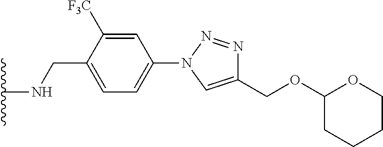 [SMILES: *NCc1ccc(-n2cc(COC3CCCCO3)nn2)cc1C(F)(F)F] |
| 110 | 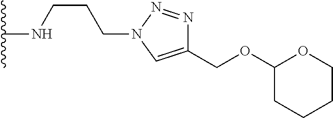 [SMILES: *NCCCn1cc(COC2CCCCO2)nn1] |
| 111 | 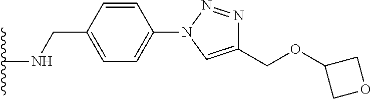 [SMILES: *NCc1ccc(-n2cc(COC3COC3)nn2)cc1] |
| 112 | 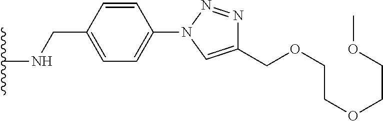 [SMILES: *NCc1ccc(-n2cc(COCCOCCOC)nn2)cc1] |
| 113 | 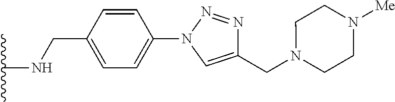 [SMILES: *NCc1ccc(-n2cc(CN3CCN(C)CC3)nn2)cc1] |
| 114 | 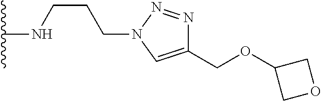 [SMILES: *NCCCn1cc(COC2COC2)nn1] |
| 115 | 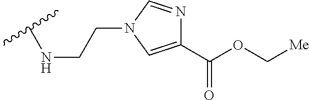 [SMILES: *NCCn1cnc(C(=O)OCC)c1] |
| 116 | 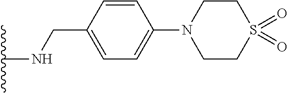 [SMILES: *NCc1ccc(N2CCS(=O)(=O)CC2)cc1] |
| 117 | 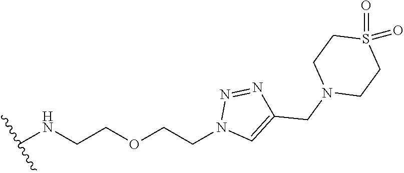 [SMILES: *NCCOCCn1cc(CN2CCS(=O)(=O)CC2)nn1] |
| 118 | 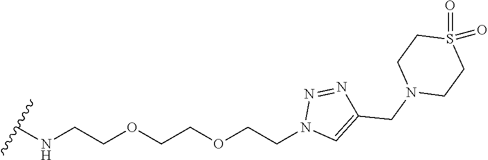 [SMILES: *NCCOCCOCCn1cc(CN2CCS(=O)(=O)CC2)nn1] |
| 119 | 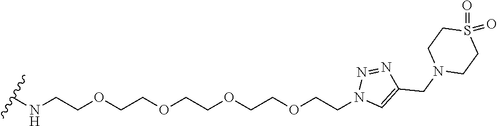 [SMILES: *NCCOCCOCCOCCOCCn1cc(CN2CCS(=O)(=O)CC2)nn1] |
| 120 | 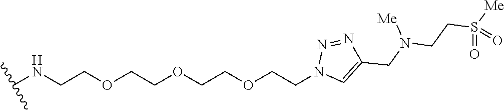 [SMILES: *NCCOCCOCCOCCn1cc(CN(C)CCS(C)(=O)=O)nn1] |
| 121 | 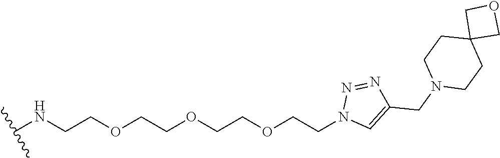 [SMILES: *NCCOCCOCCOCCn1cc(CN2CCC3(CC2)COC3)nn1] |
| 122 | 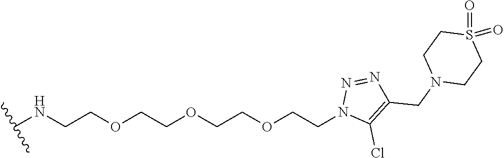 [SMILES: *NCCOCCOCCOCCn1nnc(CN2CCS(=O)(=O)CC2)c1Cl] |
| 123 | 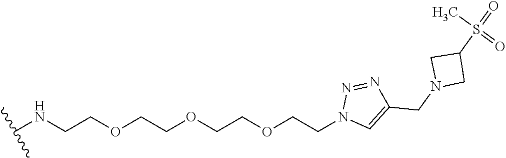 [SMILES: *NCCOCCOCCOCCn1cc(CN2CC(S(C)(=O)=O)C2)nn1] |
| 124 | 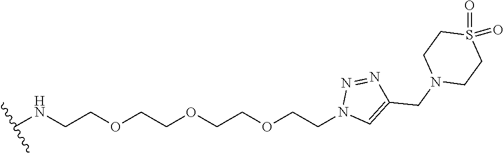 [SMILES: *NCCOCCOCCOCCn1cc(CN2CCS(=O)(=O)CC2)nn1] |
| 125 | 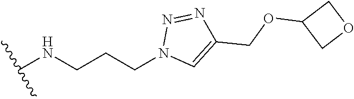 [SMILES: *NCCCn1cc(COC2COC2)nn1] |
| 126 | 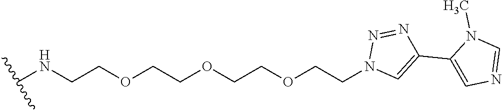 [SMILES: *NCCOCCOCCOCCn1cc(-c2cncn2C)nn1] |
| 127 | 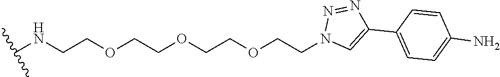 [SMILES: *NCCOCCOCCOCCn1cc(-c2ccc(N)cc2)nn1] |
| 128 | 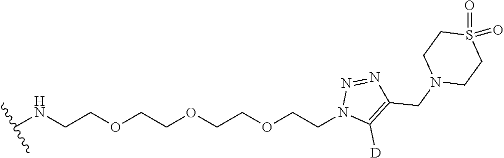 [SMILES: *NCCOCCOCCOCCn1nnc(CN2CCS(=O)(=O)CC2)c1[2H]] |
| 129 | 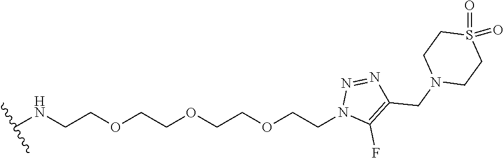 [SMILES: *NCCOCCOCCOCCn1nnc(CN2CCS(=O)(=O)CC2)c1F] |
| 130 | 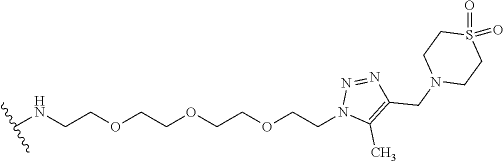 [SMILES: *NCCOCCOCCOCCn1nnc(CN2CCS(=O)(=O)CC2)c1C] |
| 131 | 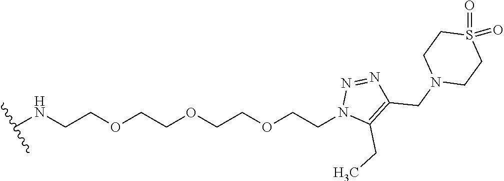 [SMILES: *NCCOCCOCCOCCn1nnc(CN2CCS(=O)(=O)CC2)c1CC] |
| 132 | 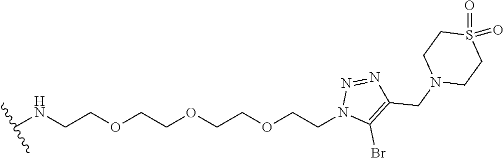 [SMILES: *NCCOCCOCCOCCn1nnc(CN2CCS(=O)(=O)CC2)c1Br] |
| 133 | 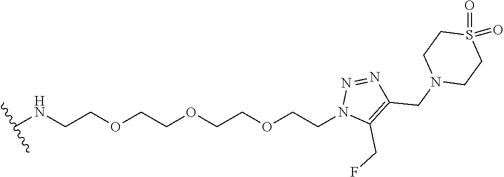 [SMILES: *NCCOCCOCCOCCn1nnc(CN2CCS(=O)(=O)CC2)c1CF] |
| 134 | 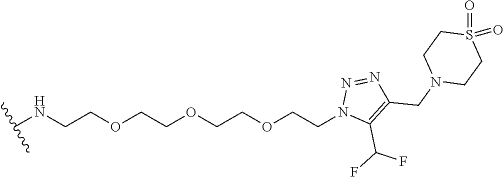 [SMILES: *NCCOCCOCCOCCn1nnc(CN2CCS(=O)(=O)CC2)c1C(F)F] |
| 135 | 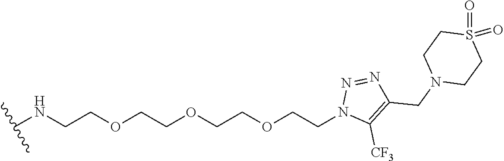 [SMILES: *NCCOCCOCCOCCn1nnc(CN2CCS(=O)(=O)CC2)c1C(F)(F)F] |
| 136 | 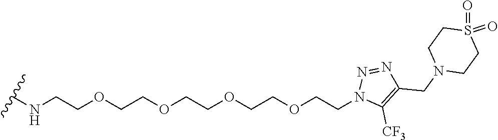 [SMILES: *NCCOCCOCCOCCOCCn1nnc(CN2CCS(=O)(=O)CC2)c1C(F)(F)F] |
| 137 | 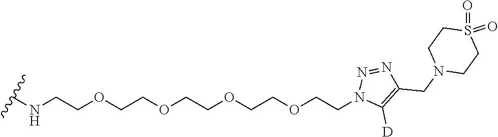 [SMILES: *NCCOCCOCCOCCOCCn1nnc(CN2CCS(=O)(=O)CC2)c1[2H]] |
| 138 | 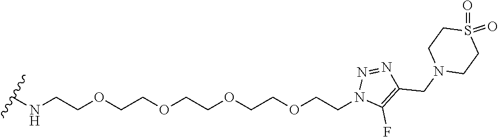 [SMILES: *NCCOCCOCCOCCOCCn1nnc(CN2CCS(=O)(=O)CC2)c1F] |
| 139 | 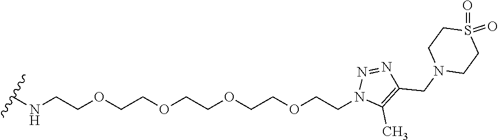 [SMILES: *NCCOCCOCCOCCOCCn1nnc(CN2CCS(=O)(=O)CC2)c1C] |
| 140 | 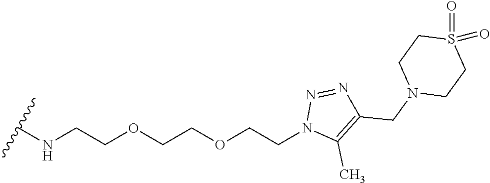 [SMILES: *NCCOCCOCCn1nnc(CN2CCS(=O)(=O)CC2)c1C] |
| 141 | 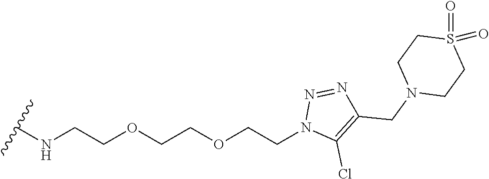 [SMILES: *NCCOCCOCCn1nnc(CN2CCS(=O)(=O)CC2)c1Cl] |
| 142 | 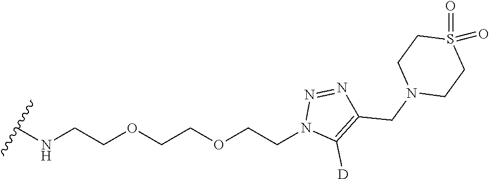 [SMILES: *NCCOCCOCCn1nnc(CN2CCS(=O)(=O)CC2)c1[2H]] |
| 143 | 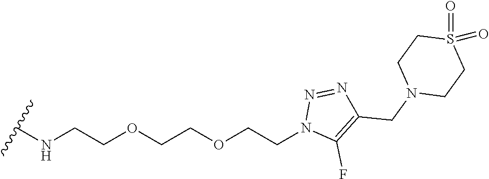 [SMILES: *NCCOCCOCCn1nnc(CN2CCS(=O)(=O)CC2)c1F] |
| 144 | 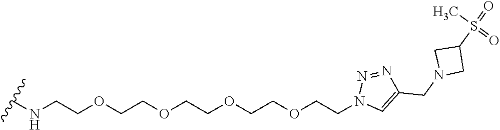 [SMILES: *NCCOCCOCCOCCOCCn1cc(CN2CC(S(C)(=O)=O)C2)nn1] |
| 145 | 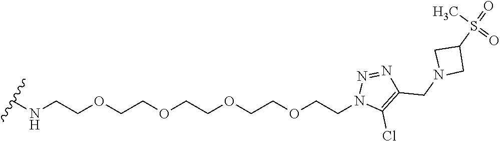 [SMILES: *NCCOCCOCCOCCOCCn1nnc(CN2CC(S(C)(=O)=O)C2)c1Cl] |
| 146 | 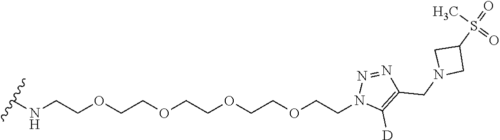 [SMILES: *NCCOCCOCCOCCOCCn1nnc(CN2CC(S(C)(=O)=O)C2)c1[2H]] |
| 147 | 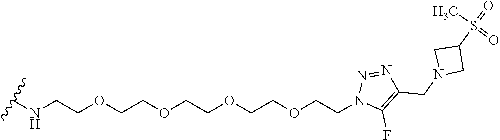 [SMILES: *NCCOCCOCCOCCOCCn1nnc(CN2CC(S(C)(=O)=O)C2)c1F] |
| 148 | 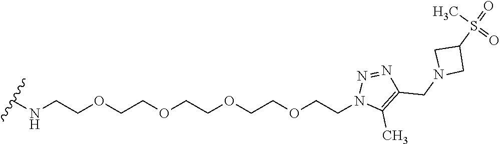 [SMILES: *NCCOCCOCCOCCOCCn1nnc(CN2CC(S(C)(=O)=O)C2)c1C] |
| 149 | 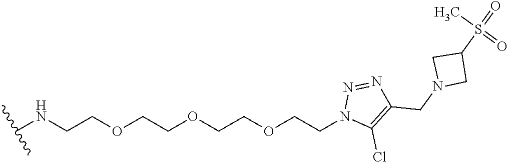 [SMILES: *NCCOCCOCCOCCn1nnc(CN2CC(S(C)(=O)=O)C2)c1Cl] |
| 150 | 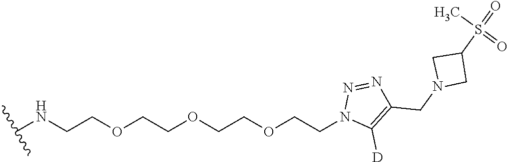 [SMILES: *NCCOCCOCCOCCn1nnc(CN2CC(S(C)(=O)=O)C2)c1[2H]] |
| 151 | 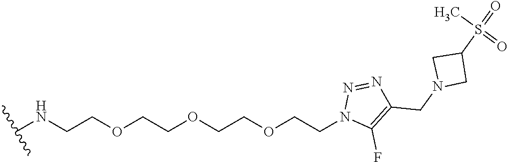 [SMILES: *NCCOCCOCCOCCn1nnc(CN2CC(S(C)(=O)=O)C2)c1F] |
| 152 | 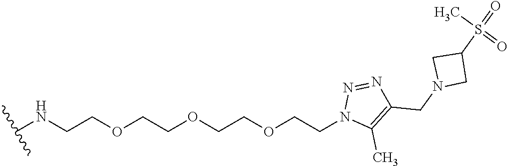 [SMILES: *NCCOCCOCCOCCn1nnc(CN2CC(S(C)(=O)=O)C2)c1C] |
| 153 | 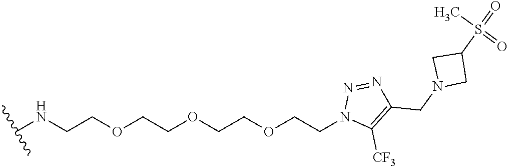 [SMILES: *NCCOCCOCCOCCn1nnc(CN2CC(S(C)(=O)=O)C2)c1C(F)(F)F] |
| 154 | 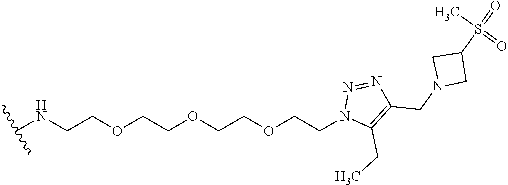 [SMILES: *NCCOCCOCCOCCn1nnc(CN2CC(S(C)(=O)=O)C2)c1CC] |

Conjugation of any of the compounds in Table 4 to a polymer (e.g., an alginate) may be performed as described in Example 2 of WO 2019/195055 or any other suitable chemical reaction.

In some embodiments, the compound is a compound of Formula (I) (e.g., Formulas (I-a), (I-b), (I-c), (I-d), (I-e), (I-f), (II), (II-a), (III), (III-a), (III-b), (III-c), or (III-d)), or a pharmaceutically acceptable salt thereof and is selected from:

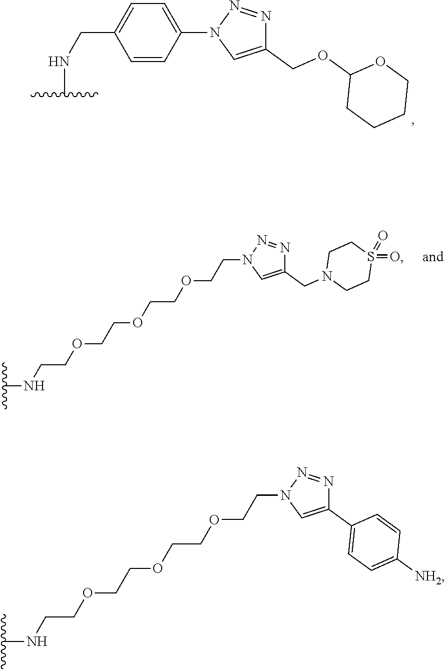

or a pharmaceutically acceptable salt thereof.

In some embodiments, the device described herein comprises the compound of

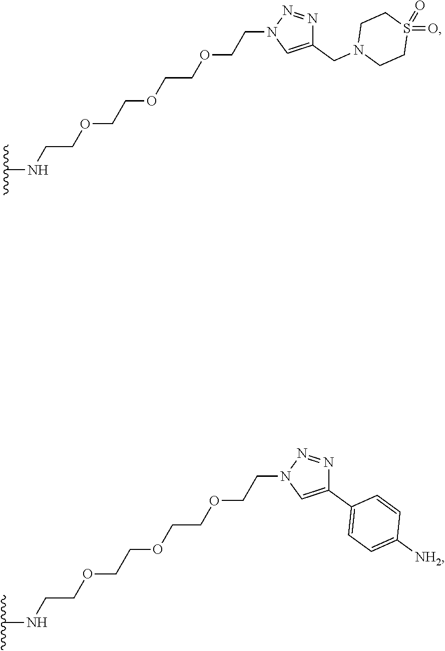 [SMILES: *NCCOCCOCCOCCn1cc(CN2CCS(=O)(=O)CC2)nn1 ; *NCCOCCOCCOCCn1cc(-c2ccc(N)cc2)nn1]

pharmaceutically acceptable salt of either compound.

In some embodiments, a compound of Formula (I) (e.g., Compound 101 in Table 4) is covalently attached to an alginate (e.g., an alginate with approximate MW<75 kDa, G:M ratio≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429.

### Glucocorticoid Compounds and Extended Release Formulations

In some embodiments, the glucocorticoid is a compound of Formula (IV):

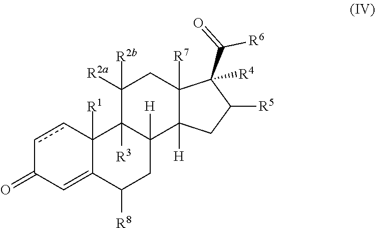

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃). In some embodiments, R⁸ is hydrogen. In some embodiments, [symbol] is a single bond. In some embodiments, [symbol] is a double bond.

In some embodiments, the glucocorticoid compound of Formula (IV) is a compound of Formula (IV-a):

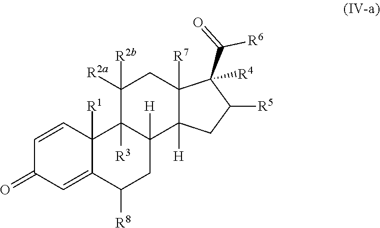

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃). In some embodiments, R⁸ is hydrogen.

In some embodiments, the glucocorticoid compound of Formula (IV) is a compound of Formula (IV-b):

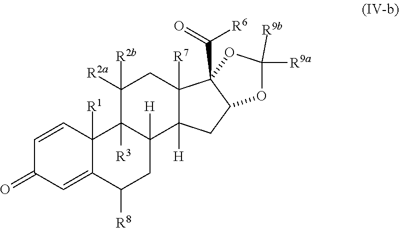

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; each of R^{9a} and R^{9b} is independently halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, each of R^{9a} and R^{9b} is independently C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl, In some embodiments, R⁶ is C₁-C₆ heteroalkyl (e.g., CH₂OH, CH₂CH₂OH). In some embodiments, R⁷ is C₁-C₆ alkyl (e.g., CH₃). In some embodiments, R⁸ is hydrogen.

In some embodiments, the glucocorticoid compound of Formula (IV) is a compound of Formula (IV-c):

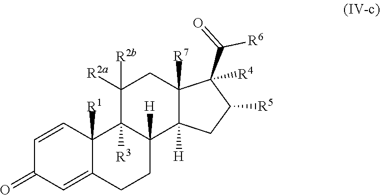

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁹ is halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃).

In some embodiments, the glucocorticoid compound of Formula (IV) is a compound of Formula (IV-d):

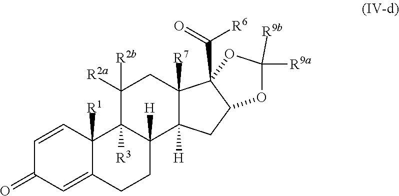

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; each of R^{9a} and R^{9b} is independently halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, each of R^{9a} and R^{9b} is independently C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl.

In some embodiments, R⁶ is C₁-C₆ heteroalkyl (e.g., CH₂OH, CH₂CH₂OH). In some embodiments, R⁷ is C₁-C₆ alkyl (e.g., CH₃).

In some embodiments, the glucocorticoid compound of Formula (IV) is a compound of Formula (IV-e):

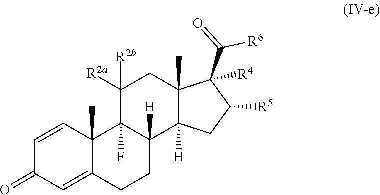

or a pharmaceutically acceptable salt, ester, hydrate, tautomer, or prodrug thereof, wherein each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁹ is halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, one of R^{2a} and R^{2b} is independently —OR^{A} and the other of R^{2a} and R^{2b} is independently hydrogen. In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH).

In some embodiments, the glucocorticoid compound of Formula (IV) is a compound of Formula-(IV-f):

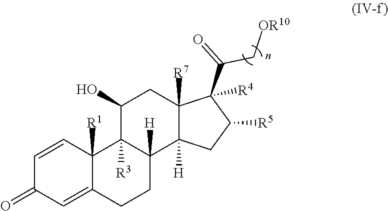

or a pharmaceutically acceptable salt, ester, hydrate, tautomer, or prodrug thereof, wherein R¹ is hydrogen, halo, or C₁-C₆ alkyl; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁹ is halo, C₁-C₆ alkyl, or —OR^{A}; R¹⁰ is hydrogen or C₁-C₆ alkyl; each of R^{A}, R^{B}, R^{C}, R^{D}, and R^{E} is independently hydrogen or C₁-C₆ alkyl; and n is 0, 1, 2, 3, or 4.

In some embodiments, R¹ is C₁-C₆ alkyl (e.g., CH₃). In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁷ is C₁-C₆ alkyl (e.g., CH₃). In some embodiments, R⁴ and R⁵ are taken together to form a 5-membered ring substituted by 2 R⁹. In some embodiments, R⁹ is C₁-C₆ alkyl (e.g., CH₃). In some embodiments, R¹⁰ is hydrogen. In some embodiments, n is 1.

In some embodiments, the glucocorticoid compound of Formula (IV) is a compound of Formula (IV-g):

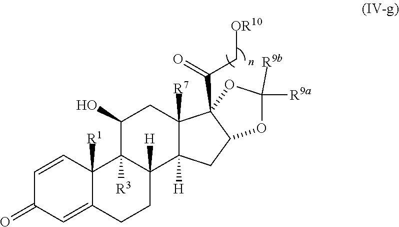

or a pharmaceutically acceptable salt, ester, hydrate, tautomer, or prodrug thereof, wherein R¹ is hydrogen, halo, or C₁-C₆ alkyl; R³ is hydrogen, halo, or C₁-C₆ alkyl; R⁷ is hydrogen, halo, or C₁-C₆ alkyl; each of R^{9a} and R^{9b} is independently halo, C₁-C₆ alkyl, or —OR^{A}; R¹⁰ is hydrogen or C₁-C₆ alkyl; each of R^{A}, R^{B}, R^{C}, R^{D}, and R^{E} is independently hydrogen or C₁-C₆ alkyl; and n is 0, 1, 2, 3, or 4.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R^{9a} and R^{9b} is independently C₁-C₆ alkyl (e.g., CH₃). In some embodiments, R⁷ is C₁-C₆ alkyl (e.g., CH₃). In some embodiments, R¹⁰ is hydrogen. In some embodiments, n is 1.

In some embodiments, the glucocorticoid compound of Formula (IV) is triamcinolone or a pharmaceutically acceptable salt, ester, hydrate, tautomer, or prodrug thereof. In some embodiments, the glucocorticoid compound of Formula (IV) is triamcinolone acetate or a pharmaceutically acceptable salt, ester, hydrate, tautomer, or prodrug thereof. In some embodiments, the glucocorticoid compound of Formula (IV) is

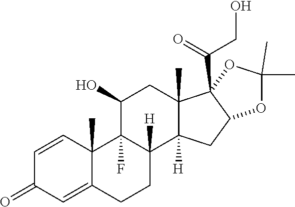

or a pharmaceutically acceptable salt, ester, hydrate, tautomer, or prodrug thereof. In some embodiments, the glucocorticoid compound of Formula (IV) is triamcinolone hexacetonide or a pharmaceutically acceptable salt, ester, hydrate, tautomer, or prodrug thereof. In some embodiments, the glucocorticoid compound of Formula (IV) is

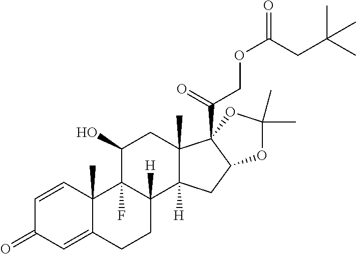

or a pharmaceutically acceptable salt, ester, hydrate, tautomer, or prodrug thereof. In some embodiments, the glucocorticoid is any of the compounds described in U.S. Pat. No. 2,990,401, which is incorporated herein by reference.

In some embodiments, the glucocorticoid compound of Formula (IV) is fluticasone or a pharmaceutically acceptable salt, ester, hydrate, tautomer, or prodrug thereof. In some embodiments, the glucocorticoid compound of Formula (IV) is fluticasone furoate or a pharmaceutically acceptable salt, ester, hydrate, tautomer, or prodrug thereof. In some embodiments, the glucocorticoid compound of Formula (IV) is

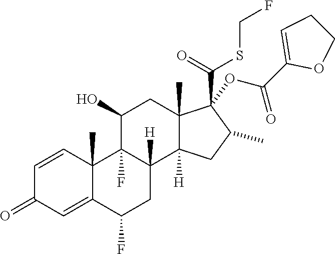

or a pharmaceutically acceptable salt, ester, hydrate, tautomer, or prodrug thereof. In some embodiments, the glucocorticoid is any of the compounds described in U.S. Pat. No. 7,101,866, which is incorporated herein by reference. In some embodiments, the glucocorticoid compound of Formula (IV) is fluticasone propionate or a pharmaceutically acceptable salt, ester, hydrate, tautomer, or prodrug thereof. In some embodiments, the glucocorticoid compound of Formula (IV) is

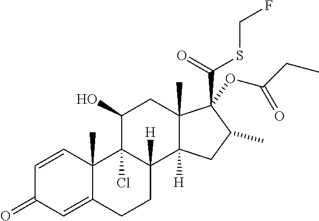

or a pharmaceutically acceptable salt, ester, hydrate, tautomer, or prodrug thereof. In some embodiments, the glucocorticoid is any of the compounds described in U.S. Pat. No. 4,335,121, which is incorporated herein by reference.

In some embodiments, the glucocorticoid compound of Formula (IV) is mometasone or a pharmaceutically acceptable salt, ester, hydrate, tautomer, or prodrug thereof. In some embodiments, the glucocorticoid compound of Formula (IV) is mometasone furoate or a pharmaceutically acceptable salt, ester, hydrate, tautomer, or prodrug thereof. In some embodiments, the glucocorticoid compound of Formula (IV) is

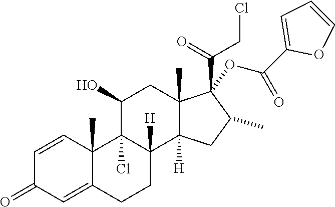

or a pharmaceutically acceptable salt, ester, hydrate, tautomer, or prodrug thereof. In some embodiments, the glucocorticoid is any of the compounds described in U.S. Pat. No. 4,472,393, which is incorporated herein by reference.

In some embodiments, the glucocorticoid compound of Formula (IV) is beclomethasone or a pharmaceutically acceptable salt, ester, hydrate, tautomer, or prodrug thereof. In some embodiments, the glucocorticoid compound of Formula (IV) is beclomethasone dipropionate or a pharmaceutically acceptable salt, ester, hydrate, tautomer, or prodrug thereof. In some embodiments, the glucocorticoid compound of Formula (IV) is

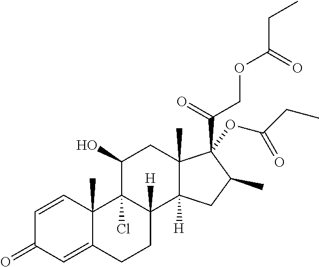

or a pharmaceutically acceptable salt, ester, hydrate, tautomer, or prodrug thereof. In some embodiments, the glucocorticoid is any of the compounds described in U.S. Pat. No. 3,645,590, which is incorporated herein by reference.

In some embodiments, the device is configured to locally release the glucocorticoid compound (e.g., as defined herein) during a desired release period after the device is implanted into a subject. In an embodiment, the desired release period is at least 30 days.

The extended release formulation can comprise a solid, semi-solid, gel, or liquid form of the glucocorticoid compound. In addition, the extended release formulation may comprise the glucocorticoid compound in conjunction with another component, such as a polymer, solvent, or other excipient. The extended release formulations described herein may provide control over glucocorticoid compound release from the device; for example, the extended release formulation may liberate a small amount of glucocorticoid compound into or out of the device over time, e.g., to provide a substantially constant concentration of the glucocorticoid compound in or around the device and or subject over an extended period of time. In an embodiment, the extended release formulation provides a dosage of the glucocorticoid compound for longer than 1 day (e.g., greater than 2 days, 3 days, 4 days, 5 days, 6 days, 1 week, 1.5 weeks, 2 weeks, 2.5 weeks, 3 weeks, 3.5 weeks, 4 weeks, 5 weeks, 6 weeks, 2 months, 3 months or more), e.g., upon administration to a subject.

In an embodiment, the extended release formulation of the glucocorticoid compound is a solid (e.g., a crystal or a granule). In an embodiment, the extended release formulation of the glucocorticoid compound is a semi-solid or a gel. In an embodiment, the extended release formulation of the glucocorticoid compound is a liquid (e.g., a solution, e.g., an aqueous solution).

The extended release formulation of the glucocorticoid compound may comprise another component, such as a polymer. Exemplary polymers may be biodegradable or non-biodegradable. In an embodiment, the polymer is a synthetic biodegradable polymer, e.g., a polymer that is not naturally occurring. In an embodiment, the polymer is a naturally occurring biodegradable polymer, e.g., a polymer that is found in nature, e.g., a polypeptide or polysaccharide. Exemplary polymers include polyesters, polyethers, polycarbonates, polyvinyl alcohols, polyurethanes, polypropylenes, polyphosphazenes, polyanhydrides, alginate, dextran, hyaluronic acid, cellulose, xanthan gum, scleroglucan, and the like.

The polymer may be linear or branched. A branched polymer may be a star polymer, comb polymer, brush polymer, dendronized polymer, ladder, or dendrimer. In an embodiment, the polymer may be cross-linked. Further, the polymer may comprise selected molecular weight ranges, degrees of polymerization, viscosities or melt flow rates. The polymer may be thermoresponsive (e.g., a gel, e.g., which becomes a solid or liquid upon exposure to heat or a certain temperature) or can also be photocrosslinkable (e.g., a gel, e.g., which becomes a solid upon photocrosslinking).

In an embodiment, the polymer in an extended release formulation of the glucocorticoid compound has a molecular weight (Mw) greater than about 1,000 Da (e.g., greater than about 2,500 Da, 5,000 Da, 7,500 Da, 10,000 Da, 12,500 Da, 15,000 Da, 20,000 Da, 25,000 Da, 50,000 Da, or more). In an embodiment, the polymer has a Mw of less than about 100,000 Da (e.g., less than about 90,000 Da, 80,000 Da, 75,000 Da, 50,000 Da, 25,000 Da, 20,000 Da, 15,000 Da, 10,000 Da, or less). In an embodiment, the polymer has a Mw of between 1,000 Da and 100,000 Da. For example, the polymer may have a Mw between 1,000 Da and 50,000 Da, 2,500 Da and 40,000 Da, 5,000 Da and 30,000 Da, 5,000 Da and 20,000 Da, or 10,000 Da and 20,000. In an embodiment, the polymer has a Mw between 1,000 Da and 50,000 Da. In an embodiment, the polymer has a Mw between 5,000 Da and 20,000 Da.

The polymer may comprise any type of end group on its termini. For example, the polymer may comprise an amine, ester, acid, hydroxyl, acyl, amide, or ether end group on one or more of its termini. In an embodiment, the polymer comprises the same end group on each termini. In an embodiment, the polymer comprises a plurality of end groups on its termini (e.g., at least 2, 3, 4, 5 different end groups on its termini). In an embodiment, the polymer comprises an acid group on at least one of its termini. In an embodiment, the polymer comprises an ester group on at least one of its termini.

In an embodiment, the polymer in an extended release formulation of the glucocorticoid compound is a polyester. Polyesters degrade through the nonspecific hydrolysis of their ester bonds. Exemplary polyesters include poly(lactic acid) (PLA), poly(L-lactic acid) (PLLA), poly(D-lactic acid) (PDLA), poly(glycolic acid) (PGA), poly(lactic glycolic acid) (PLGA), poly(caprolactone) (PCL), and poly(lactic caprolactone) (PLCL). In an embodiment, the polyester polymer comprises lactic acid (e.g., a lactide). In an embodiment, the polyester polymer comprises a glycolic acid (e.g., a glycolide). In an embodiment, the polyester polymer comprises poly(lactic glycolic acid) (PLGA) (e.g., poly(D,L-lactic glycolic acid), poly(D-lactic glycolic acid), or poly(L-lactic glycolic acid)). The PLGA may be obtained from any commercial supplier or may be prepared de novo. In an embodiment, the PLGA is a Resomer® PLGA.

The polymer may be a homogeneous polymer or a blended polymer (e.g., a co-polymer of a block co-polymer). In the case of blended polymers, the polymer may comprise a plurality of monomer units (e.g., at least 2, 3, 4, 5, or 6 types of monomer units). In an embodiment, for blended polymers comprising 2 monomer units (Unit A and Unit B), the ratio of Unit A to Unit B is between 0.1:99.1 to 99.1:0.1. For example, the ratio of Unit A to Unit B may be 0.1:99.1, 0.5:99.5, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, 99:1, 99.5:0.5, or 99.1:0.1. Similarly, a blended polymer may comprise 3 monomer units (Unit A, Unit B, and Unit C) with exemplary ratios of Unit A to Unit B to Unit C between 1:1:99 to 99:1:1 (including varying amounts of Unit B).

In an embodiment, the polymer in an extended release formulation of the glucocorticoid compound has a viscosity (e.g., inherent viscosity) of between 0.01 dL/g and 2.5 dL/g. For example, the polymer may have a viscosity of between 0.05 dL/g and 2.0 dL/g, 0.1 dL/g to 0.5 dL/g, 0.1 dL/g to 0.3 dL/g, or 1.0 dL/g and 1.5 dL/g. In an embodiment, the polymer is free of a contaminant, such as a metal, catalyst, free radical, oxygen, microbe, or endotoxin. In an embodiment, the polymer is sterile. In an embodiment, the polymer is provided as a powder, granules, pellets, crystals, a gel, or a liquid.

The extended release formulation of the glucocorticoid compound may comprise another component, such as a solvent. The solvent may be an organic solvent that is miscible with water, or an organic solvent that is not miscible with water. In an embodiment, the solvent is an organic solvent that aids in the solubility and stability of the glucocorticoid compound over time. In an embodiment, the organic solvent is any organic solvent approved by the Food and Drug Administration (FDA) for use in humans. Exemplary organic solvents include dimethylamide (DMA), dimethylformamide (DMF), N-methyl-2-pyrrolidone (NMP), dimethylsulfoxide (DMSO), formamide, tetrahydrofuran (THF), ethanol, isopropanol, acetone, ethyl acetate, 1,4-dioxane, pyridine, and triethylamine (TEA). In an embodiment, the organic solvent comprises an amine (e.g., a primary amine, secondary amine, or tertiary amine). In an embodiment, the organic solvent comprises a ketone. In an embodiment, the organic solvent has a boiling point greater than about 100° C., e.g., greater than about 150° C., 200° C., 250° C., 300° C. or higher.

Exemplary extended release formulations described herein may comprise a both a polymer and an organic solvent, e.g., at a fixed ratio of polymer to organic solvent. Factors such as solubility, stability, toxicity, or volume of the final composition are often taken into consideration when determining the ratio of the polymer with the organic solvent. In one embodiment, the ratio of the polymer to the organic solvent is between 50:50 and 90:10 (w/w) (e.g., 50:50, 60:40, 70:30, 80:20, or 90:10). In another embodiment, the ratio of the polymer to the organic solvent is between 10:90 and 50:50 (w/w) (e.g., 10:90, 20:80, 30:70, 40:60, or 50:50).

The concentration of glucocorticoid compound in the extended release formulation may be within the range of 0.01% to 50% w/w (e.g., about 0.1% to 40% w/w or 1% to 25% w/w). In some embodiments, the concentration of glucocorticoid compound in the extended release formulation is about 0.1% to 25% w/w, e.g., between about 0.1% and 20%, about 0.1% and 15%, about 0.1% and 10%, about 0.5% and 25%, about 0.5% and 20%, about 0.5% and 15%, about 0.5% and 10%, about 1% and 25%, about 1% and 20%, about 1% and 15%, or about 1% and 10% w/w). In some embodiments, the concentration of glucocorticoid compound in the extended release formulation is about 0.05%, about 0.1%, about 0.5%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11%, about 11.5%, about 12%, about 12.5%, about 13%, about 13.5%, about 14%, about 14.5%, about 15%, about 14.5%, about 20%, or about 25% w/w.

The concentration of glucocorticoid compound in the extended release formulation may be within the range of 0.01% to 50% w/v (e.g., about 0.1% to 40% w/v or 1% to 25% w/v). In some embodiments, the concentration of glucocorticoid compound in the extended release formulation is about 0.1% to 25% w/v, e.g., between about 0.1% and 20%, about 0.1% and 15%, about 0.1% and 10%, about 0.5% and 25%, about 0.5% and 20%, about 0.5% and 15%, about 0.5% and 10%, about 1% and 25%, about 1% and 20%, about 1% and 15%, or about 1% and 10% w/v). In some embodiments, the concentration of glucocorticoid compound in the extended release formulation is about 0.05%, about 0.1%, about 0.5%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11%, about 11.5%, about 12%, about 12.5%, about 13%, about 13.5%, about 14%, about 14.5%, about 15%, about 17.5%, about 20%, or about 25% w/v.

The extended release formulations compositions provided are formulated for sustained or delayed release of a glucocorticoid compound described herein (e.g., a compound of Formula (I)). Sustained release, as described herein, refers to the property of a composition by which release of a glucocorticoid compound from the extended release formulation occurs over an extended period of time as compared to the release from an isotonic saline solution. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the glucocorticoid compound or any other material associated with the sustained release formulation. To illustrate further, a wide range of degradation rates or release rates may be obtained by adjusting the features of the extended release formulation, such as the identity of the polymer or any related feature of the polymer (including the molecular weight, viscosity, end group, etc.), the identity of the organic solvent, the concentration of any one of the polymer, organic solvent, or glucocorticoid compound, or ratios thereof.

One protocol generally accepted in the field that may be used to determine the release rate of the glucocorticoid compound from an extended release formulation described herein entails disposing the extended release formulation into a physiological environment and removing samples of the mixture and various time points for analysis, e.g., by HPLC.

The release rate of the glucocorticoid compound from the extended release formulation may also be characterized by the amount of glucocorticoid compound released per day per quantity of polymer present in the sustained release formulation. For example, in some embodiments, the release rate may vary from about 1 ng or less of the glucocorticoid compound per day per quantity of polymer to about 500 or more ng/day/quantity of polymer. Alternatively, the release rate may be about 0.05, 0.5, 5, 10, 25, 50, 75, 100, 125, 150, 175, 200, 250, 300, 350, 400, 450, or 500 ng/day/quantity of polymer. In still other embodiments, the release rate of the glucocorticoid compound may be 10,000 ng/day/ng/day/quantity of polymer, or even higher.

In an embodiment, the release of glucocorticoid compound from an extended release formulation described herein may be described as the half-life (i.e., T_{1/2}) of such material in the formulation.

In an embodiment, an extended release formulation described herein provides a dosage of the glucocorticoid compound for greater than 1 day (e.g., greater than 2 days, 3 days, 4 days, 5 days, 6 days, 1 week, 1.5 weeks, 2 weeks, 2.5 weeks, 3 weeks, 3.5 weeks, 4 weeks, 5 weeks, 6 weeks, 2 months, 3 months or more), e.g., upon administration of the device to a subject. In an embodiment, the extended release formulation provides a dosage of the glucocorticoid compound for greater than 1 week (e.g., greater than 1.5 weeks, 2 weeks, 2.5 weeks, 3 weeks, 3.5 weeks, 4 weeks, 5 weeks, 6 weeks, 2 months, 3 months or more), e.g., upon administration to a subject. In an embodiment, the extended release formulation provides a dosage of the glucocorticoid compound between 1 week and 10 weeks (e.g., 1 week and 8 weeks, 1 week and 6 weeks, 1 week and 4 weeks, 2 weeks and 8 weeks, 3 weeks and 6 weeks), e.g., upon administration to a subject. In an embodiment, the extended release formulation provides a dosage of the glucocorticoid compound between 0.5 ug and 500 ug per day (e.g., between 1 ug and 500 ug, 1 ug and 250 ug, 1 ug and 100 ug, 1 ug and 50 ug, 5 ug and 500 ug, 5 ug and 250 ug, 5 ug and 100 ug, 5 ug and 50 ug, 10 ug and 250 ug, 10 ug and 100 ug, 10 ug and 50 ug, 50 ug and 500 ug, 50 ug and 250 ug, 50 ug and 100 ug).

In an embodiment, the extended release formulation has the property that it forms a drug depot within a device described herein. A drug depot refers to a localized mass of a particular glucocorticoid compound (e.g., solid particles of a glucocorticoid described herein), wherein the glucocorticoid compound is gradually released from the device, e.g., by diffusion. A drug depot may comprise at least one region which provides a bolus of the glucocorticoid compound, and a remaining region which provides a sustained release of the glucocorticoid compound.

In an embodiment, the extended release formulation comprises a plurality of particles of the glucocorticoid compound suspended in a hydrogel in the device, e.g., within a hydrogel that forms the outer compartment or the inner, cell-containing compartment of a two-compartment device described herein. In an embodiment, the particles comprise the glucocorticoid compound in crystalline form.

In an embodiment, the glucocorticoid compound particles are prepared by adding a desired quantity of an amorphous powder of the glucocorticoid compound (e.g., a glucocorticoid) to a desired volume of a solution comprising a hydrogel-forming polymer (e.g., an alginate or alginate mixture described herein), sonicating the resulting powder-polymer mixture until a substantially homogenous suspension is formed, and contacting droplets of the resulting suspension with a cross-linking solution. In an embodiment, the quantity of the glucocorticoid powder and the volume of the polymer solution are selected to achieve a mixture of 2.5 mg to 5.0 mg powder per mL polymer solution. In an embodiment, the genetically-modified cells are added to the homogenous suspension and the resulting cell-containing suspension is used to form the inner compartment of a two-compartment hydrogel capsule as described herein. In an embodiment, the glucocorticoid is triamcinolone hexacetonide, fluticasone furoate, fluticasone propionate or mometasone furoate.

### Genetically Modified Cells

Devices of the present disclosure contain (e.g., encapsulate) cells genetically modified to express and secrete at least one therapeutic substance (e.g., a protein) that has a biological activity or biological effect useful for treating a subject in need of therapy with the substance. The cells may be in the form of single cells, or provided in another form, e.g., disposed on a microcarrier (e.g., a bead or matrix) or as one or more three-dimensional aggregates of cells (e.g., one or more cell clusters).

The genetically modified cell(s) may be derived from a variety of different cell types (e.g., human cells), including adipose cells, epidermal cells, epithelial cells, endothelial cells, fibroblast cells, islet cells, mesenchymal stem cells, keratinocyte cells, pericytes, subtypes of any of the foregoing, cells derived from any of the foregoing, cells derived from induced pluripotent stem cells and mixtures of any of the foregoing. Exemplary cell types include the cell types recited in WO 2017/075631. In some embodiments, the cells are derived from a cell-line shown in Table 3 below.

TABLE 3
Exemplary cell lines

| | | Germ | |
|---|---|---|---|
| Cell Line | Cell Type | Layer | Commercial Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841- | Epithelial (Colon) | Endoderm | ATCC (CRL-1790) |
| CoN | | | |
| HaCat | Keratinocyte | Ectoderm | Addexbio (T0020001) |
| HHSEC | Endothelial (Hepatic Sinusoidal) | Endoderm | Sciencellonline.com (#5000) |
| Huv-EC-C | Endothelial (Embryonic umbilical) | Mesoderm | ATCC (CRL-1730) |
| MCF-10A | Epithelial (Mammary Gland) | Ectoderm | ATCC (CRL-10317) |
| MRC-5 | Fibroblast (Lung) | Mesoderm | ATCC (CCL-171) |
| MSC, human | Mesenchyme (Bone Marrow) | Mesoderm | ATCC (PCS-500-012) |
| MSC, mouse | Mesenchyme (Bone Marrow) | Mesoderm | Cyagen (MU BMX-01001) |
| WS-1 | Fibroblast (Skin) | Ectoderm | ATCC (CRL-1502) |
| 293F | Epithelial (Embryonic Kidney) | Mesoderm | Thermo Fisher (R790007) |

In some embodiments, the device does not comprise any islet cells or any cells capable of producing insulin in a glucose-responsive manner. In an embodiment, cells contained in a device of the disclosure, e.g., genetically modified RPE cells, have one or more of the following characteristics: (i) are not capable of producing insulin (e.g., insulin A-chain, insulin B-chain, or proinsulin) in an amount effective to treat diabetes or another disease or condition that may be treated with insulin; (ii) not capable of producing insulin in a glucose-responsive manner; or (iii) not derived from an induced pluripotent stem cell that was engineered or differentiated into insulin-producing pancreatic beta cells.

Cells may be genetically modified to express and secrete therapeutic peptides and proteins using any of a variety of genetic engineering techniques known in the art. For example, a cell may be transfected with an expression vector comprising an exogenous nucleotide sequence(s) encoding a desired polypeptide operably linked to control elements necessary or useful for gene expression, such as promoters, ribosomal binding sites, enhancers, and polyA signal. Typically, the exogenous sequence encodes a precursor form of the polypeptide, e.g., includes a secretory signal sequence. In some embodiments, the signal sequence in a precursor polypeptide consists essentially of an amino acid sequence shown in Table 4 below. In an embodiment, the signal sequence is MGWRAAGALLLALLLHGRLLA (SEQ TD NO:13).

TABLE 4
Exemplary secretory signal peptide sequences

| Source Protein | Amino Acid Sequence |
|---|---|
| Albumin | MKWVTFISLLFLFSSAYS |
| | (SEQ ID NO: 8) |
| Kappa Leader | MVLQTQVFISLLLWISGAYG |
| | (SEQ ID NO: 9) |
| Plasminogen | MQMSPALTCLVLGLALVFGEGSA |
| activator | (SEQ ID NO: 10) |
| inhibitor 1 | |
| Thrombospondin-1 | MGLAWGLGVLFLMHVCGT |
| | (SEQ ID NO: 11) |
| Fibronectin | MLRGPGPGLLLLAVQCLGTAVPSTGASK |
| | SKR (SEQ ID NO: 12) |
| Basement membrane- | MGWRAAGALLLALLLHGRLLA |
| specific heparan | (SEQ ID NO: 13) |
| sulfate proteo- | |
| glycan core | |
| protein (HSPG2) | |
| Agrin | MAGRSHPGPLRPLLPLLVVAACVLPGAG |
| | G (SEQ ID NO: 14) |
| H7 Leader | MEFGLSWVFLVALFRGVQC (SEQ ID |
| | NO: 15) |
| L2 Leader | MKYLLPTAAAGLLLLAAQPAMA (SEQ |
| | ID NO: 16) |
| HMM34 | MRPTWAWWLFLVLLLALWAPARG |
| | (SEQ ID NO: 17) |
| HMM38 | MWWRLWWLLLLLLLLWPMVWA (SEQ |
| | ID NO: 18) |
| Gaussia luciferase | MGVKVLFALICIAVAEA (SEQ ID |
| | NO: 19) |
| Alpha-1-antitrypsin | MPSSVSWGILLLAGLCCLVPVSLA |
| | (SEQ ID NO: 20) |
| Interleukin-10 | MHSSALLCCLVLLTGVRA (SEQ ID |
| | NO: 21) |

When engineering cells to co-express two or more therapeutic peptides or proteins, a multicistronic vector may be employed. In some embodiments, a genetically modified cell described herein comprises an exogenous nucleotide sequence, which encodes a therapeutic protein, stably inserted into one or more genomic locations (e.g., open chromatin region(s)).

In some embodiments, the encapsulated cells are genetically modified with a regulatable expression system, to allow controlled expression of the therapeutic substance(s). A variety of such systems are known in the art and include, for example: kill switches (see, e.g., Wu, C. et al., Mol. Ther. Methods Clin Dev. 2014; 1: 14053); On/Off systems (see, e.g., Gossen, M and Gujar, H., Proc. Natl. Acad. Sci. USA, Vol 89, pp. 5547-5551 (1992); Liberles, S., et al., Proc. Natl. Acad. Sci. USA, Vol. 94, pp. 7825-7830 (1997); feed forward and negative feedback systems (Lillacci, G. et al., Nuc. Acids Res, Vol 46, Issue 18 (12 Oct. 2018) pp. 9855-9863); temperature inducible systems (Miller, I. et al., ACS Synth Biol. 2018; 7(4): 1167-1173).

In some embodiments, a genetically modified cell described herein is derived from an ARPE-19 cell, and may express and secrete any of the therapeutic peptides and proteins described herein (or combinations of such peptides and proteins).

### Therapeutic Substances

In an embodiment, the therapeutic substance is for the prevention or treatment of a disease, disorder, or condition, e.g., those described in international application publications WO 2017/075631 A1 and WO 2020/069429 A1. The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule. Exemplary therapeutic agents include the agents listed in WO 2017/075631 and WO 2020/069429 A1.

In some embodiments, the therapeutic agent is a peptide or polypeptide (e.g., a protein), such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by an engineered cell can have a naturally occurring amino acid sequence, or may contain a variant of the naturally occurring sequence. The variant can be a naturally occurring or non-naturally occurring amino acid substitution, mutation, deletion or addition relative to the reference sequence, e.g., a naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence.

The therapeutic peptide or polypeptide may be expressed as part of a fusion protein, which comprises a first amino acid sequence which forms a therapeutic domain (e.g., has substantially the same activity as the therapeutic peptide or polypeptide) and a second amino acid sequence with a desired property. In an embodiment, the second amino acid sequence facilitates secretion of the fusion protein by the encapsulated cells, e.g., a signal peptide sequence from a heterologous protein. In an embodiment, the second amino acid sequence forms a tissue-targeting moiety, e.g., binds to a tissue-specific protein of interest, e.g., a blood-brain barrier transporter, liver, a tumor. In an embodiment, the second amino acid sequence forms a half-life-extending domain, e.g., an immunoglobulin Fc, albumin, a serum albumin binding domain. In an embodiment, the fusion protein comprises both a tissue-targeting domain and a half-life extending domain in addition to the therapeutic domain.

In some embodiments, the protein is a clotting factor or a coagulation factor, e.g., a blood clotting factor or a blood coagulation factor. Non-limiting examples of blood clotting factors, and exogenous coding sequences, are described in WO 2019/067766. In an embodiment, the protein is a FVII protein, and comprises an amino acid sequence described in WO 2020/198695.

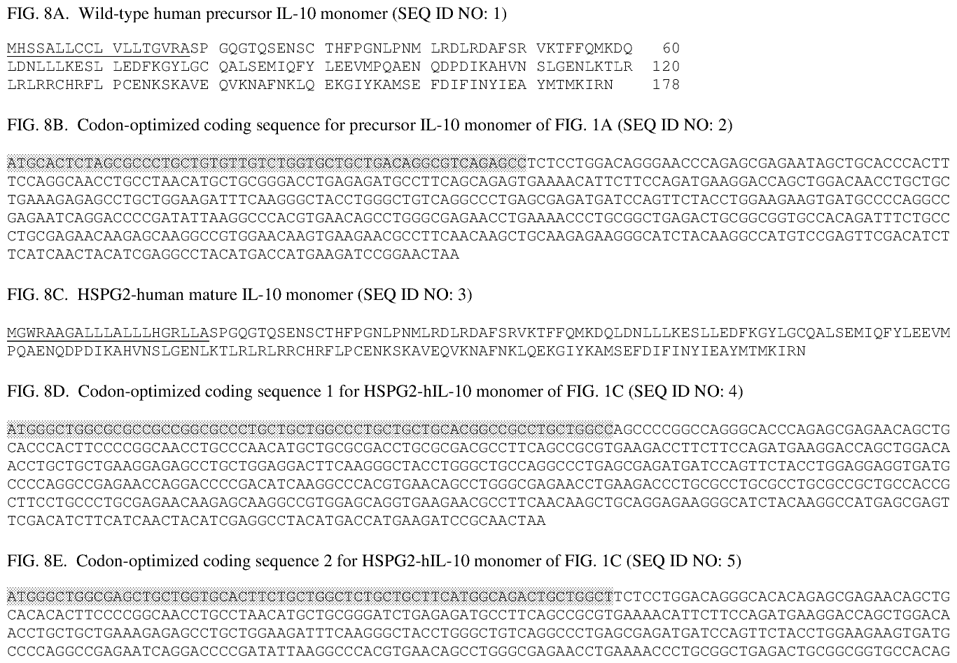

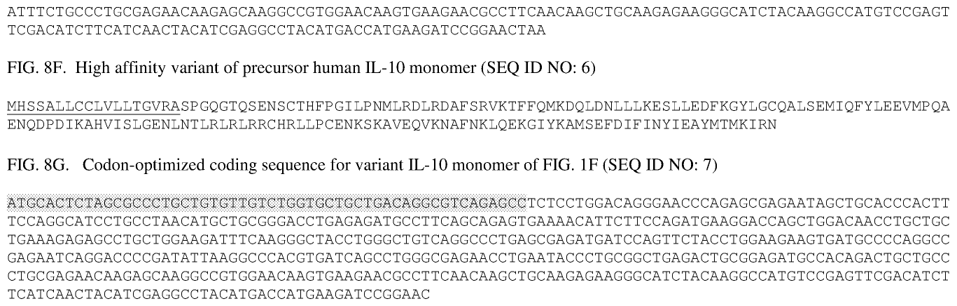

In some embodiments, the protein is a cytokine or a cytokine receptor, or a chimeric protein including cytokines or their receptors. In an embodiment, the protein is an IL-10 protein. In an embodiment, the IL-10 protein comprises an amino acid sequence shown in FIG. 8 (e.g., FIG. 8A, FIG. 8C or FIG. 8F). In an embodiment, the IL-10 protein is encoded by a codon-optimized sequence shown in FIG. 8 (e.g., FIG. 8B, FIG. 8D or FIG. 8G).

In some embodiments, the replacement therapy or replacement protein is an enzyme. In an embodiment, the enzyme is an alpha-galactosidase A (GLA) protein and comprises an amino acid sequence described in WO 2020/198685. In an embodiment, the therapeutic protein is alpha-L-iduronidase (IDUA), glucocerebrosidase, N-sulfoglucosamine sulfohydrolase (SGSH), N-acetyl-alpha-d-glucosaminidase, N-acetylglucosamine 4-sulphatase, urate oxidase, phenyalanine hydroxylase, or phenylalanine ammonia lyase.

### Preparing Two-Compartment Hydrogel Capsules

Each compartment of a device described herein may comprise an unmodified polymer, a polymer modified with a compound of Formula (I), or a blend thereof. Briefly, to prepare a device configured as a two-compartment hydrogel capsule, a volume of a first polymer solution (e.g., comprising an unmodified polymer, a polymer modified with a compound of Formula (I), or a blend thereof, and optionally containing cells,) is loaded into a first syringe connected to the inner lumen of a coaxial needle. The first syringe may then be connected to a syringe pump oriented vertically above a vessel containing an aqueous cross-linking solution which comprises a cross-linking agent, a buffer, and an osmolarity-adjusting agent. A volume of the second polymer solution (e.g., comprising an unmodified polymer, a polymer modified with a compound of Formula (I), or a blend thereof, and optionally containing cells) is loaded into a second syringe connected to the outer lumen of the coaxial needle. The second syringe may then be connected to a syringe pump oriented horizontally with respect to the vessel containing the cross-linking solution. A high voltage power generator may then be connected to the top and bottom of the needle. The syringe pumps and power generator can then be used to extrude the first and second polymer solutions through the syringes with settings determined to achieve a desired droplet rate of polymer solution into the cross-linking solution. The skilled artisan may readily determine various combinations of needle lumen sizes, voltage range, flow rates, droplet rate and drop distance to create 2-compartment hydrogel capsule compositions in which the majority (e.g., at least 80%, 85%, 90% or more) of the capsules are within 10% of the target size and desired shape. After exhausting the first and second volumes of polymer solution, the droplets may be allowed to cross-link in the cross-linking solution for certain amount of time, e.g., about five minutes.

An exemplary process for preparing a composition of millicapsules (e.g., 1.5 mm diameter) is described in WO 2019/195055.

### Device Compositions

A device described herein may be provided as a preparation or composition for implantation or administration to a subject, e.g., a human subject. In some embodiments, a device preparation or composition comprises at least 2, 4, 8, 16, 32, 64 or more devices, and at least 20%, 30%, 35%, 40%, 45%, 50%, 55%, 60%, 65%, 70%, 75%, 80%, 85%, 90%, 95% or 100% of the devices in the preparation or composition have a characteristic as described herein, e.g., mean diameter or mean pore size or cell density.

A device composition may be configured for implantation, or implanted or disposed into or onto any site of the body. In some embodiments, a device, preparation or composition is configured for implantation, implanted, or disposed into the subcutaneous fat of a subject, or into the muscle tissue of a subject. In some embodiments, the device, device preparation or device composition is configured for implantation or implanted or disposed into the peritoneal cavity (e.g., the omentum). In some embodiments, the device is configured for implantation or implanted or disposed into or onto the lesser sac, also known as the omental bursa or bursalis omentum. The lesser sac refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). In some embodiments, the lesser sac comprises a high concentration of adipose tissue. A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, preparation or composition into the omentum (e.g., the lesser sac) are provided in M. Pellicciaro et al. (2017) CellR4 5(3):e2410.

In some embodiments, a device or preparation is easily retrievable from a subject, e.g., without causing injury to the subject or without causing significant disruption of the surrounding tissue. In an embodiment, the device or preparation can be retrieved with minimal or no surgical separation of the device(s) from surrounding tissue, e.g., via minimally invasive surgical approach, extraction, or resection.

A device, composition or preparation can be configured to provide continuous delivery of an immunomodulatory protein for a variety of time periods after implant into a mammalian recipient (e.g., a human patient), including: a short continuous delivery (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less) or prolonged delivery (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or longer).

In some embodiments, the device composition or preparation does not contain any capsule, device, implant or other object disclosed in any of WO2012/112982, WO2012/167223, WO2014/153126, WO2016/019391, WO2016/187225, WO 2018/232027, WO 2019/068059, WO 2019/169089, US2012-0213708, US 2016-0030359, and US 2016-0030360.

### Methods of Treatment

Described herein are methods for preventing or treating a disease, disorder, or condition in a subject through administration or implantation of a device, a device preparation or a device composition described herein. In some embodiments, the methods described herein directly or indirectly reduce or alleviate at least one symptom of a disease, disorder, or condition. In some embodiments, the methods described herein prevent or slow the onset of a disease, disorder, or condition. In some embodiments, the subject is a human.

In some embodiments, the disease, disorder, or condition affects a system of the body, e.g. the nervous system (e.g., peripheral or central nervous system), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes (Type 1 or Type 2), a heart disease, an autoimmune disease, a cancer, a liver disease, a lysosomal storage disease, a blood clotting disorder or a coagulation disorder, an orthopedic condition, an amino acid metabolism disorder.

In some embodiments, the disease, disorder or condition is a neurodegenerative disease. Exemplary neurodegenerative diseases include Alzheimer's disease, Huntington's disease, Parkinson's disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down's syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick's disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, mesothelioma and lung cancers, brain cancer (e.g., glioblastoma), sarcoma, ovarian cancer, pancreatic cancer, renal cancer, liver cancer, testicular cancer, prostate cancer, uterine cancer and metastatic peritoneal cancers.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Hurler syndrome (also known as mucopolysaccharidosis type I (MPS-1)), Hunter syndrome (also known as MPS-2), lysosomal acid lipase deficiency, Niemann-Pick disease, Salla disease, Sanfilippo syndrome (also known as mucopolysaccharidosis type IIIA (MPS-3A)), multiple sulfatase deficiency, Maroteaux-Lamy syndrome, metachromatic leukodystrophy, Krabbe disease, Scheie syndrome, Hurler-Scheie syndrome, Sly syndrome (MPS-7), hyaluronidase deficiency, Pompe disease, Danon disease, gangliosidosis, MPS-6 and Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand disease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is not Type I diabetes and/or is not Type II diabetes.

The present invention further comprises methods for identifying a subject having or suspected of having a disease, disorder, or condition described herein, and upon such identification, administering to the subject a device, device preparation or device composition described herein. In an embodiment, the subject is a human.

### ENUMERATED EXEMPLARY EMBODIMENTS

1. An implantable device comprising: (a) at least one cell-containing compartment comprising a living cell or a first plurality of living cells, optionally wherein the cell-containing compartment is surrounded by a barrier compartment; and (b) an extended release formulation of a glucocorticoid compound, wherein the extended release formulation and device are configured to provide continuous, local release of the glucocorticoid compound from the device during a release period (e.g., at least 7 days) after implantation into an immune-competent subject.
2. The implantable device of any one of claims 1 to 14, wherein the glucocorticoid compound is a compound of Formula (IV):

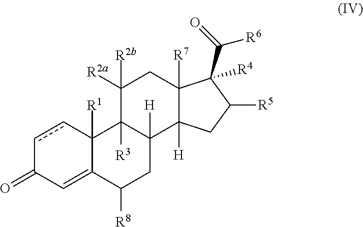

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.
3. The implantable device of embodiment 1 or 2, wherein the glucocorticoid compound is selected from the group consisting of triamcinolone hexacetonide, triamcinolone acetonide, fluticasone furoate, fluticasone propionate, mometasone furoate, and beclomethasone diproprionate.
4. The implantable device of any one of embodiments 1 to 3, wherein the glucocorticoid compound is triamcinolone hexacetonide (TAH).
5. The implantable device of any one of embodiments 1 to 3, wherein the glucocorticoid compound is fluticasone furoate (FF).
6. The implantable device of any one of embodiments 1 to 3, wherein the glucocorticoid compound is mometasone furoate (MF).
7. The implantable device of any one of embodiments 1 to 6, wherein the device comprises at least one of the following features:
(i) the glucocorticoid compound is released from the implanted device in an amount effective to inhibit PFO formation on the device during the entire release period;
(ii) an afibrotic compound (e.g., as defined herein) disposed on the exterior surface of the device;
(iii) the exterior surface of the device does not comprise any alginate;
(iv) all of the cells in the plurality of living cells are mammalian cells genetically modified to express and secrete a first therapeutic substance;
(v) a second plurality of living mammalian cells genetically modified to express and secrete a second therapeutic substance that is different than the first substance;
(vi) each therapeutic substance secreted by the living cells is a protein comprising a heterologous signal peptide sequence, optionally the signal peptide sequence consists essentially of MGWRAAGALLLALLLHGRLLA (SEQ ID NO:13);
(vii) the plurality of living cells are derived from ARPE-19 cells;
(viii) the plurality of living cells are derived from a non-human mammalian cell line; and
(ix) the plurality of living cells are derived from an induced pluripotent stem cell line.
8. The device of any one of embodiments 1 to 7, wherein the release period is at least any of 10 days, 15 days, 30 days, 60 days, or 90 days.
9. The implantable device of embodiment 7 or 8, which comprises feature (i).
10. The implantable device of any one of embodiments 7 to 9, which comprises feature (ii).
11. The implantable device of embodiment 10, wherein the cell-containing compartment is surrounded by the barrier compartment and the barrier compartment comprises the exterior surface.
12. The implantable device of embodiment 10 or 11, wherein the exterior surface comprises a polymer and the afibrotic compound is covalently attached to the first polymer.
13. The implantable device of embodiment 12, wherein the polymer is an alginate, optionally wherein the alginate has an approximate molecular weight <75 kDa and a G:M ratio ≥1.5.
14. The implantable device of embodiment 12, wherein the exterior surface does not comprise any alginate.
15. The implantable device of any one of embodiments 10 to 14, wherein the afibrotic compound is a compound of Formula (I):

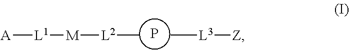

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²
L² is a bond;
M is absent, alkyl, heteroalkyl, cycloalkyl, heterocyclyl, aryl, or heteroaryl, each of which is optionally substituted by one or more R³
P is absent, cycloalkyl, heterocycyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.
16. The implantable device of embodiment 16, wherein the afibrotic compound is selected from the compounds shown in Table 2 or a pharmaceutically acceptable salt of the selected compound.
17. The implantable device of embodiment 16, wherein the afibrotic compound is selected from the group consisting of Compounds 100, 101, 102, 114, 122 and 123 shown in Table 4 above, or a pharmaceutically acceptable salt of the selected compound.
18. The implantable device of embodiment 16, wherein the afibrotic compound is:

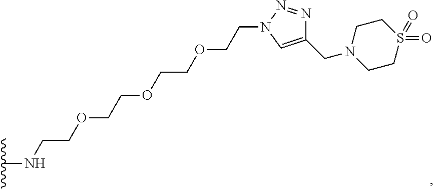 [SMILES: *NCCOCCOCCOCCn1cc(CN2CCS(=O)(=O)CC2)nn1]

or a pharmaceutically acceptable salt thereof.
19. The implantable device of embodiment 16, wherein the afibrotic compound is:

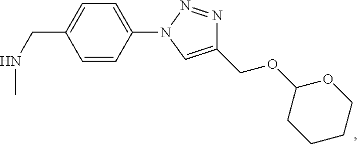 [SMILES: CNCc1ccc(-n2cc(COC3CCCCO3)nn2)cc1]

or a pharmaceutically acceptable salt thereof
20. The implantable device of embodiment 16, wherein the afibrotic compound is Compound 122 or a pharmaceutically acceptable salt thereof.
21. The implantable device of embodiment 16, wherein the afibrotic compound is Compound 123 or a pharmaceutically acceptable salt thereof.
22. The implantable device of any one of embodiments 7 to 21, which comprises feature (iv) and optionally feature (vi).
23. The implantable device of any one of embodiments 7 to 21, which comprises feature (iv) and (v) and optionally feature (vi).
24. The implantable device of any one of embodiments 7 to 23, wherein the first therapeutic substance comprises a therapeutic peptide or therapeutic protein.
25. The implantable device of any one of embodiments 7 to 24, wherein the first therapeutic protein therapeutic protein is an antibody, a cytokine, a soluble cytokine receptor, an enzyme, a hormone, a coagulation factor or a blood clotting factor.
26. The implantable device of any one of embodiments 7 to 24, wherein the first therapeutic protein is a human IL-10 protein, a human GLA protein or a human IDUA protein.
27. The implantable device of any one of embodiments 7 to 24, wherein the first therapeutic protein is an enzyme that is deficient or missing in a lysosomal storage disorder (LSD).
28. The implantable device of any one of embodiments 7 to 27, which comprises feature (vii).
29. The implantable device of any one of embodiments 7 to 27, which comprises feature (viii).
30. The implantable device of any one of embodiments 7 to 27, which comprises feature (ix).
31. The implantable device of any one of embodiments 1 to 30, wherein the cell-containing compartment comprises a hydrogel-forming polymer.
32. The implantable device of any one of embodiments 1 to 31, wherein the hydrogel-forming polymer comprises an alginate, optionally wherein the alginate has an approximate molecular weight of 75 kDa to 150 kDa.
33. The implantable device of embodiment 31 or 32, wherein the hydrogel-forming polymer is covalently modified with a cell-binding peptide, optionally wherein the cell-binding peptide is GRGDSP.
34. The implantable device of any one of embodiments 7 to 33, wherein the device is a hydrogel capsule comprising the cell-containing compartment surrounded by the barrier compartment.
35. The implantable device of embodiment 34, wherein the barrier compartment comprises a cross-linked hydrogel.
36. The implantable device of embodiment 34 or 35, wherein the hydrogel capsule has a spherical shape and has a diameter of 0.5 millimeter to 5 millimeters, optionally a diameter of about 1 mm to about 2 mm, or about 1.3 mm to about 1.7 mm or about 1.5 mm.
37. The implantable device of any one of the above embodiments, wherein the extended release formulation of the glucocorticoid compound is a sustained release formulation.
38. The implantable device of any one of embodiments 1 to 37, wherein the extended release formulation of the glucocorticoid compound is a controlled release formulation.
39. The implantable device of any one of the above embodiments, wherein all of the genetically modified cells in the device are derived from ARPE-19 cells.
40. The implantable device of any one of embodiments 1 to 39, wherein all of the genetically modified cells in the device are derived from induced pluripotent stem cells.
41. A device composition comprising a preparation of devices and a pharmaceutically acceptable excipient, wherein each device in the preparation is a device as defined in any of the above embodiments, optionally wherein the composition has a volume of less than 10 milliliters, less than 8 ml, or less than 5 ml.
42. A method of providing a therapeutic substance to a subject, comprising administering to the subject the device of any one of embodiments 7 to 41 or the device composition of embodiment 42.
43. A genetically modified cell comprising an exogenous nucleotide coding sequence shown in FIG. 8B, 8D, 8E or 8G.
44. The genetically modified cell of embodiment 44, which is derived from ARPE-19 cells.

### EXAMPLES

In order that the disclosure described herein may be more fully understood, the following examples are set forth. The examples described in this application are offered to illustrate the devices (e.g., capsules, particles, chemical modifications, compositions and methods provided herein and are not to be construed in any way as limiting their scope.

### Example 1: Culturing of Exemplary Genetically-Modified ARPE-19 Cells for Encapsulation

Genetically modified ARPE-19 cells expressing one or more therapeutic substances described herein may be cultured to produce a composition of cells suitable for encapsulation in two compartment hydrogel capsules. The genetically-modified cells are grown in complete growth medium (DMEM:F12 with 10% FBS) in 150 cm² cell culture flasks or CellSTACK® Culture Chambers (Corning Inc., Corning, NY). To passage cells, the medium in the culture flask are aspirated, and the cell layer is briefly rinsed with phosphate buffered saline (pH 7.4, 137 mM NaCl, 2.7 mM KCl, 8 mM Na₂HPO₄, and 2 mM KH₂PO₄, Gibco). 5-10 mL of 0.05% (w/v) trypsin/0.53 mM EDTA solution (“TrypsinEDTA”) is added to the flask, and the cells are observed under an inverted microscope until the cell layer is dispersed, usually between 3-5 minutes. To avoid clumping, cells are handled with care and hitting or shaking the flask during the dispersion period is minimized. If the cells do not detach, the flasks are placed at 37° C. to facilitate dispersal. Once the cells disperse, 10 mL complete growth medium is added and the cells are aspirated by gentle pipetting. The cell suspension is transferred to a centrifuge tube and spun down at approximately 125×g for 5-10 minutes to remove TrypsinEDTA. The supernatant is discarded, and the cells are resuspended in fresh growth medium. Appropriate aliquots of cell suspension are added to new culture vessels, which are incubated at 37° C. The medium is renewed weekly.

### Example 2: Preparation of Exemplary Modified Polymers

Chemically-modified Polymer. A polymeric material may be chemically modified with a compound of Formula (I) (or pharmaceutically acceptable salt thereof) prior to formation of a device described herein (e.g., a hydrogel capsule). For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest (e.g., Compound 101 shown in Table 2) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a solution of low molecular weight alginate, chemically modified with a medium conjugation density of Compound 101 (CM-LMW-Alg-101-Medium polymer), the dissolved unmodified low molecular weight alginate (approximate MW<75 kDa, G:M ratio≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a solution of low molecular weight alginate, chemically modified with a high conjugation density of Compound 101 (CM-LMW-Alg-101-High polymer), the dissolved unmodified low-molecular weight alginate (approximate MW<75 kDa, G:M ratio ≥1.5) is treated with 2-chloro-4,6-dimethoxy-1,3,5-triazine (5.1 mmol/g alginate) and N-methylmorpholine (10.2 mmol/g alginate) and Compound 101 (10.5 mmol/g alginate).

The reaction is warmed to 55° C. for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

CBP-Alginates. A polymeric material may be covalently modified with a cell-binding peptide prior to formation of a device described herein (e.g., a hydrogel capsule described herein) using methods known in the art, see, e.g., Jeon O, et al., Tissue Eng Part A. 16:2915-2925 (2010) and Rowley, J. A. et al., Biomaterials 20:45-53 (1999).

For example, in the case of alginate, an alginate solution (1%, w/v) is prepared with 50 mM of 2-(N-morpholino)-ethanesulfonic acid hydrate buffer solution containing 0.5M NaCl at pH 6.5, and sequentially mixed with N-hydroxysuccinimide and 1-ethyl-3-[3-(dimethylamino)propyl]carbodiimide (EDC). The molar ratio of N-hydroxysuccinimide to EDC is 0.5:1.0. The peptide of interest is added to the alginate solution. The amounts of peptide and coupling reagent added depends on the desired concentration of the peptide bound to the alginate, e.g., peptide conjugation density. By increasing the amount of peptide and coupling reagent, higher conjugation density can be obtained. After reacting for 24 h, the reaction is purified by dialysis against ultrapure deionized water (diH2O) (MWCO 3500) for 3 days, treated with activated charcoal for 30 min, filtered (0.22 mm filter), and concentrated to the desired viscosity.

The conjugation density of a peptide-modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

### Example 3: Preparation of Exemplary Alginate Solutions for Making Hydrogel Capsules

70:30 mixture of chemically-modified and unmodified alginate. A low molecular weight alginate (PRONOVA™ VLVG alginate, NovaMatrix, Sandvika, Norway, cat. #4200506, approximate molecular weight <75 kDa; G:M ratio≥1.5) is chemically modified with Compound 101 to produce chemically modified low molecular weight alginate (CM-LMW-Alg-101) solution with a viscosity of 25 cp to 35 cP and a conjugation density of 5.1% to 8% N, as determined by combustion analysis for percent nitrogen. A solution of high molecular weight unmodified alginate (U-HMW-Alg) is prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution is blended with the U-HMW-Alg solution at a volume ratio of 70% CM-LMW-Alg to 30% U-HMW-Alg (referred to herein as a 70:30 CM-Alg:UM-Alg solution).

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Alginate Solution Comprising Cell Binding Sites. A solution of SLG20 alginate is modified with a peptide consisting of GRGDSP as described above and concentrated to a viscosity of about 100 cP. The amount of the peptide and coupling reagent used are selected to achieve a target peptide conjugation density of about 0.2 to 0.3, as measured by combustion analysis.

### Example 4: Preparation of Exemplary Glucocorticoid Particles Suspended in Alginate Solutions Using Indirect Sonication

An amount of a solid, amorphous form of the desired glucocorticoid compound (e.g., TAH) is added to an alginate solution (e.g., GRGDSP-medium molecular weight alginate in saline, viscosity of about 100 cP) in a conical tube (e.g., 15 mL to 50 mL) in an amount sufficient to form a suspension with a desired concentration of the solid immunosuppressant in the alginate solution (e.g., 1 mg to 10 mg compound per mL alginate solution). The tube is placed in a 5.7 liter sonication bath (Ultrasonic Cleaner, VWR Catalog No. 97043-940, Input: 117V˜60 Hz, Operating frequency: 35 kHz) at room temperature for 3 minutes to 10 minutes, removed and vortexed for one minute at 3,000 RPM (Thermo Scientific™ LP Vortex Mixer). The sonication and vortex steps are repeated until no visible powder was observed, indicating that a fine, substantially homogenous suspension has been generated. The suspension is kept at 4° C. until use and is used within 6 hours.

### Example 5: Formation of Exemplary Two-Compartment Hydrogel Capsules

Genetically modified cells, and optionally glucocorticoid particles, are encapsulated in two-compartment hydrogel capsules according to the protocol described below.

Prior to fabricating hydrogel capsules, buffers and alginate solutions are sterilized by filtration through a 0.2-μm filter using aseptic processes.

Immediately before encapsulation, a desired volume of a composition comprising the cells (e.g., from a culture of the cells as described in Example 1) is centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells ae centrifuged again and all of the supernatant is aspirated. The cell pellet is resuspended in the GRGDSP-modified alginate solution described in Example 3 or in Example 4 at a desired cell density (e.g., about 50 to 150 million suspended single cells per ml alginate solution).

To prepare two-compartment hydrogel millicapsules of about 1.5 mm diameter an electrostatic droplet generator is set up as follows: an ES series 0-100-kV, 20-watt high-voltage power generator (EQ series, Matsusada, NC, USA) is connected to the top and bottom of a coaxial needle. For capsules without an immunosuppressant, a suitable needle has an inner lumen of 22G, outer lumen of 18G, Ramé-Hart Instrument Co., Succasunna, NJ, USA. To prepare capsules that co-encapsulate immunosuppressant particles in the inner compartment, the inner lumen of the coaxial needle may need to have a larger diameter to avoid needle clogging by the immunosuppressant particles, e.g., a useful coaxial needle has an inner lumen of 21G and an outer lumen of 17G, Ramé-Hart Instrument Co., Succasunna, NJ, USA).

The inner lumen is attached to a first 5-ml Luer-lock syringe (BD, NJ, USA), which is connected to a syringe pump (Pump 11 Pico Plus, Harvard Apparatus, Holliston, MA, USA) that is oriented vertically. The outer lumen is connected via a luer coupling to a second 5-ml Luer-lock syringe which is connected to a second syringe pump (Pump 11 Pico Plus) that is oriented horizontally. A first alginate solution containing the genetically modified cells (as single cells) suspended in a GRGDSP-modified alginate solution is placed in the first syringe and a cell-free alginate solution comprising a mixture of a chemically-modified alginate and unmodified alginate (e.g., 70:30 mixture described in Example 3) is placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump are 12.06 mm diameter and the flow rates of each pump are adjusted to achieve a flow rate ratio of 1:1 for the two alginate solutions. Thus, with the total flow rate set at 10 ml/h, the flow rate for each alginate solution was about 5 mL/h. Control (empty) capsules are prepared in the same manner except that the alginate solution used for the inner compartment is a cell-free solution.

After extrusion of the desired volumes of alginate solutions, the alginate droplets are crosslinked for five minutes in a cross-linking solution which contained 25 mM HEPES buffer, 20 mM BaCl₂, 0.2M mannitol and 0.01% of poloxamer 188. Capsules that fall to the bottom of the crosslinking vessel are collected by pipetting into a conical tube. After the capsules settle in the tube, the crosslinking buffer is removed, and capsules are washed four times in HEPES buffer, two times in 0.9% saline, and two times in culture media and stored in an incubator at 37° C.

All of the spheres described in Examples 6-11 were two-compartment hydrogel capsules prepared substantially as described in this Example 5.

### Example 6: Evaluating Diffusion of Glucocorticoid from Capsules Containing Encapsulated Glucocorticoid Particles

A glucocorticoid particle suspension is prepared as described in Example 4 using amounts of the test glucocorticoid (solid, amorphous form) and alginate solution to achieve a concentration of 2 mg of the glucocorticoid solid/mL alginate solution. Two-compartment hydrogel capsules containing the suspension in the inner compartment are prepared as in Example 5, except that cells are not typically included. Multiple capsules (e.g., five) are placed in each of a desired number of replicate wells (e.g., four) in a multiwell tissue culture plate (e.g., Falcon® 12-well Clear Flat Bottom Not Treated Multiwell Cell Culture Plate). Each of the replicate wells contains 1 mL of a cell culture media (e.g., DMEMF12, Gibco Cat. No. 113 30-032 with rFBS, Gibco Cat. No. 26140-095). The media in each well is replaced with fresh 1 mL of the media at day 1, day 4 and day 7. Pictures of each well are taken using a BZX Fluorescence Microscope at day 1, day 4, day 7 to visually assess the capsules for the presence of encapsulated glucocorticoid particles.

The quantity of glucocorticoid diffusing from the capsules into the media over time is estimated by removing an aliquot (e.g., 20 μL) of the media supernatant from each replicate well on multiple days (e.g., day 1, day 4 and day 7) and diluting the removed aliquot 10-fold with Methanol (e.g., 180 μL). The sample is vortexed briefly and centrifuged at 12,500×g for 10 minutes. The supernatant is removed and transferred to an LCMS vial for analysis. The samples are analyzed using a Thermo Fisher Vanquish UPLC interfaced to a Thermo Fisher Q Exactive mass spectrometer. The chromatographic separation is performed on a 50 mm×2.1 mm Waters BEH C18 chromatography column with 1.7 μm particle size. Mobile phases A and B are 0.1-% aqueous formic acid and 0.1% formic acid in acetonitrile, respectively. The column temperature is held at 65° C. and the mobile phase is flowed at 500 μL/min. Injection volume is 5 μL. The separation is accomplished with a gradient started at 10% mobile phase B and increasing to 99% over 2.5 minutes. The column is held at 99% B for 1 minute before returning to initial conditions. The initial conditions (10% B) are held for 1.5 minutes before the next LC injection. The Q Exactive mass spectrometer is configured with an electrospray ionization source operating in positive ion mode. Data are acquired using a full scan method scanning from 400-1250 m/z at a resolution of 70,000. Data are analyzed by extracting a 10 ppm window centered on the +H ions for each glucocorticoid of interest. The Retention Time for TAH is 2.41 minutes and m/z is 533.2909.

A suspension of particles of a test glucocorticoid compound prepared as in Example 4 is likely to provide the desired extended release of the glucocorticoid compound from the capsules if less than 10% of the encapsulated amount of the glucocorticoid has been released at day 7, and preferably less than <5%, <2%, or <1% has been released at day 7.

### Example 7. Continuous Release of Soluble Drug from Alginate Encapsulated Solvent-Free Drug Particles

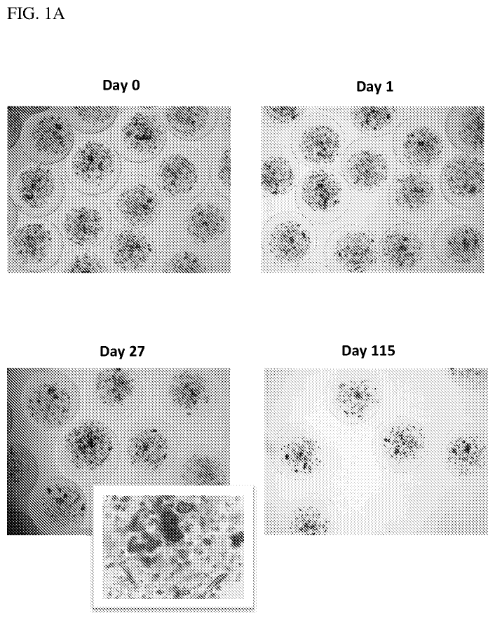

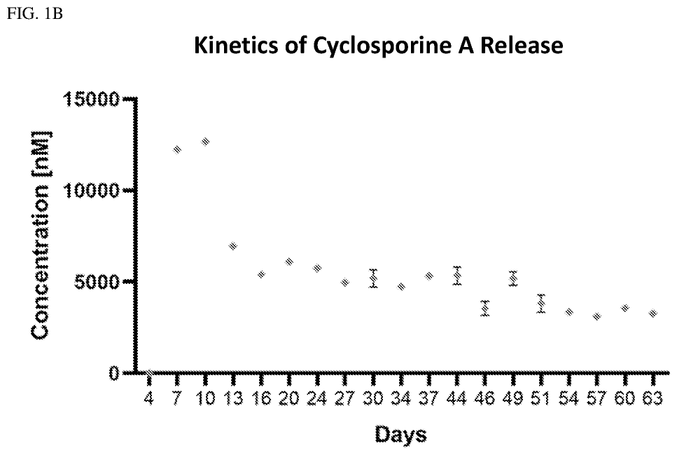

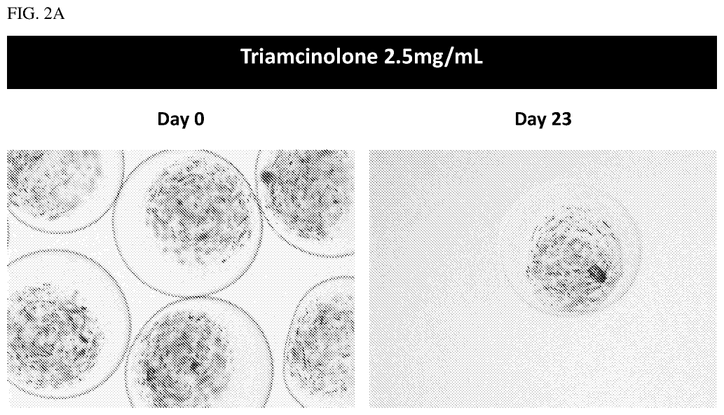

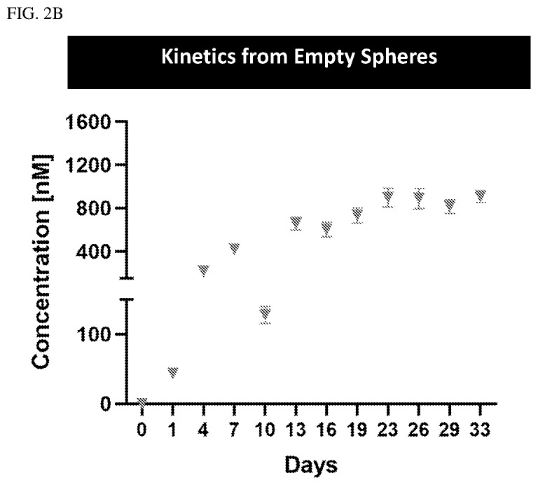

Cyclosporine A particles and Triamcinolone Hexacetonide (TAH) particles were encapsulated in the inner compartment of two compartment alginate hydrogel capsules (“spheres”). The bright field micrographs in FIG. 1A and FIG. 2A show the presence of Cyclosporine A particles and TAH particles exclusively in the inner compartment. Barium-crosslinked alginate spheres containing the drug particles were incubated in 1 ml of Normosol at 37 C and soluble Cyclosporine A and TAH released from the spheres into the Normosol solution over a 62 and 32 day period respectively was measured and the results are shown in FIG. 1B and FIG. 2B. These data demonstrate efficient encapsulation and extended release of the drugs from the alginate spheres.

### Example 8. Co-Encapsulation of Genetically Modified Human Cells and Drug Particles

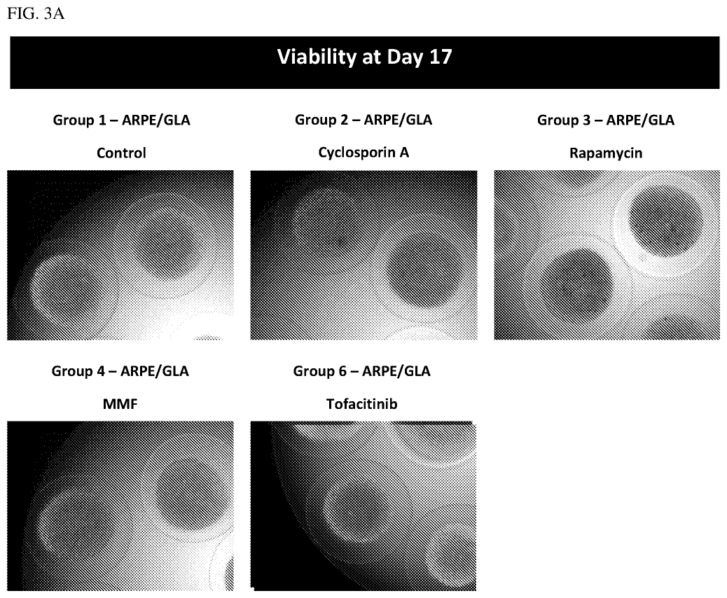

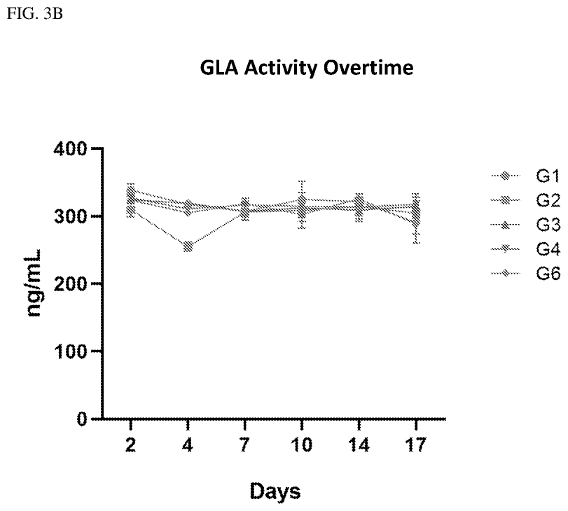

ARPE19 cells genetically modified to express human alpha-galactosidase (GLA) (hereinafter GLA-secreting human cells) were co-encapsulated with drug particles in the inner compartment of two-compartment alginate spheres. The fluorescent micrographs in FIG. 3A show the viability of live cells marked by Calcein-AM (Green) staining was unaffected. The graph in FIG. 3B demonstrates the production of GLA by cells co-encapsulated with particles of the indicated drug is maintained over the course of 18 days of in vitro culture.

### Example 9. Co-Encapsulation of Particles of Cyclosporin a, Rapamycin, MMF or Tofactitinib do not Prevent PFO Induction on Alginate Spheres Containing Xenogeneic Cells

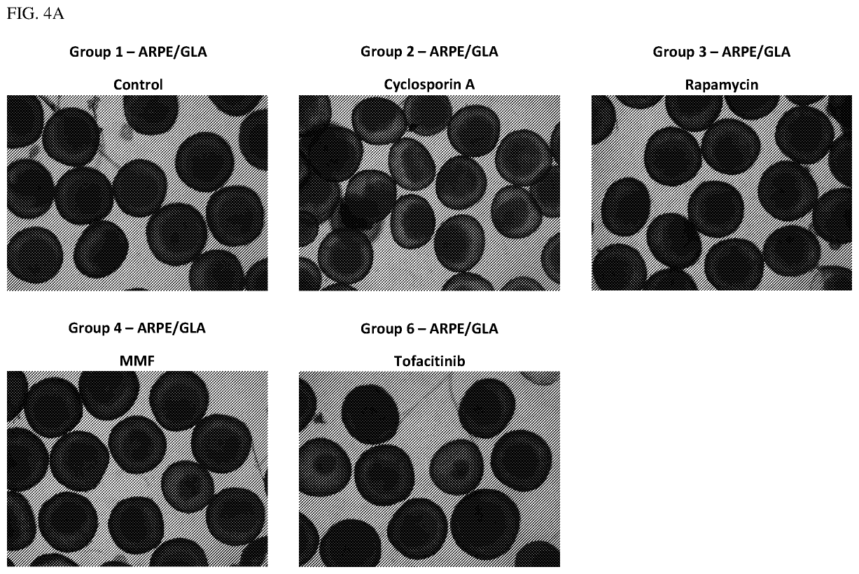

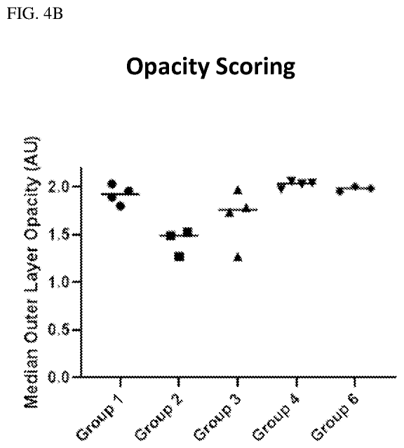

This experiment evaluated the ability of co-encapsulation of Cyclosporin A, Rapamycin, MMF or Tofactitinib particles to prevent PFO induction on alginate spheres in a xenogeneic setting. GLA-secreting human cells were co-encapsulated with particles of the indicated drug and implanted in the peritoneum of immune-competent C57Bl/6 mice. Spheres explanted after 20 days exhibited extensive PFO induction as depicted by opaque staining in the brightfield micrographs shown in FIG. 4A. Unbiased computer aided opacity scoring (FIG. 4B) revealed no statistically significant decrease in PFO induction in any of the experimental groups.

### Example 10. Co-Encapsulation of TAH, FP, FF or MF Prevents PFO Induction on Alginate Spheres Containing Xenogeneic Cells

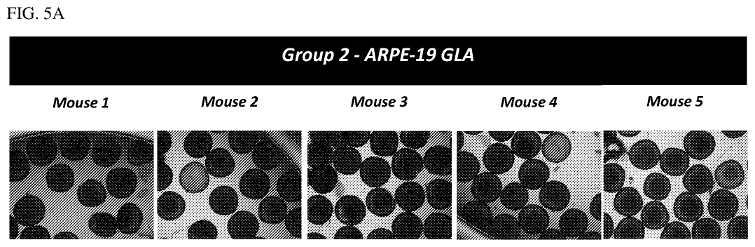

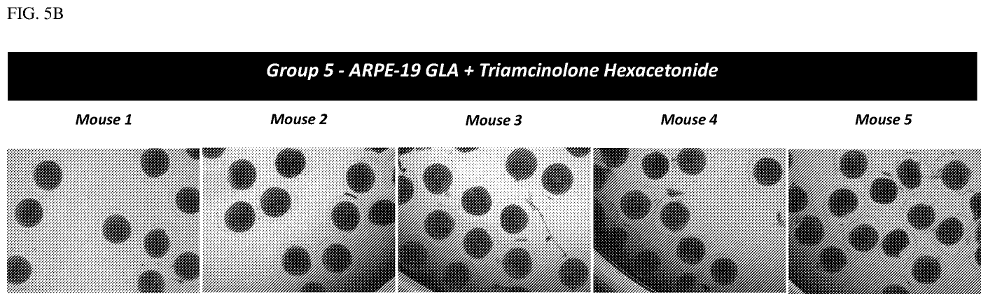

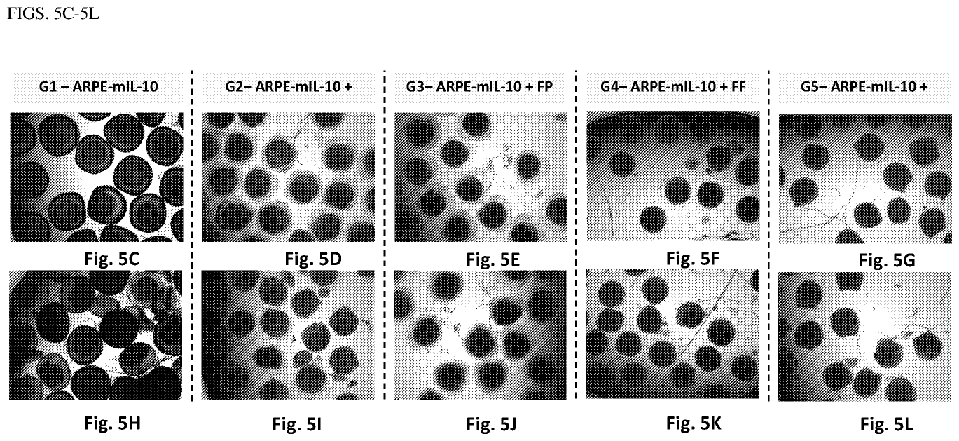

GLA-secreting human ARPE-19 cells were co-encapsulated with or without TAH particles into spheres that were then implanted in the peritoneum of immune-competent C57Bl/6 mice. Spheres without TAH particles explanted (retrieved) after 20 days exhibited extensive PFO induction as depicted by opaque staining in the brightfield micrographs shown in FIG. 5A. In contrast co-encapsulation of these cells with TAH particles completely prevented induction of PFO as exhibited by non-opaque and clear retrieved spheres at 20 days post-implant (FIG. 5B).

In a second experiment, spheres containing ARPE-19 cells secreting murine IL-10 (mIL-10) co-encapsulated with or without glucocorticoid particles (TAH, FP, FF or MF) were implanted in the peritoneum of five groups of immune-competent C57Bl/6 mice (five mice per group). At 28 days, spheres were explanted and examined for PFO. As shown in FIGS. 5C-5L, which shows representative images from two mice in each group, extensive PFO was present on spheres without glucocorticoid (left panel) but minimal to no PFO was observed on spheres containing particles of one of the glucocorticoids.

In a third experiment, spheres co-encapsulating FF particles and either unmodified human ARPE-19 cells or ARPE-19 cells genetically modified to secrete one of two different fusion proteins comprising human parathyroid hormone were implanted in the peritoneum of immune-competent Sprague Dawley rats (five rats per group). At 21 days, explanted spheres from each of the three groups exhibited minimal to no PFO (data not shown).

### Example 11. Co-Encapsulation of TAH does not Affect Viability of GLA-Secreting Cells and Preserves Therapeutic Protein Production In Vivo

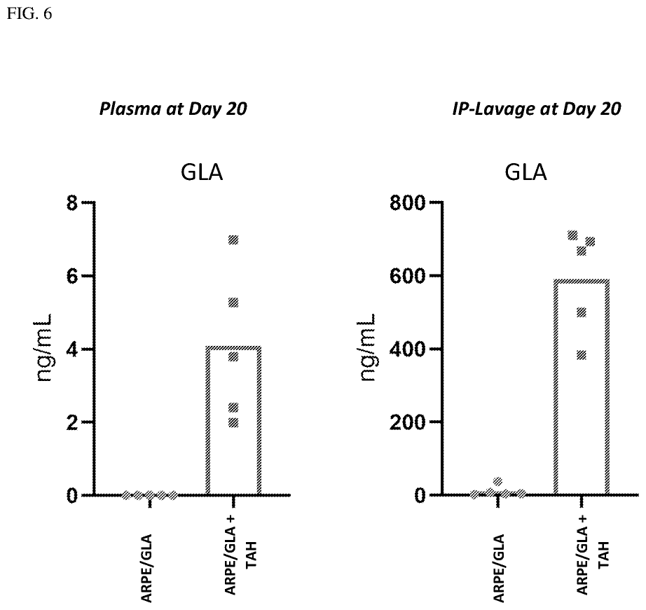

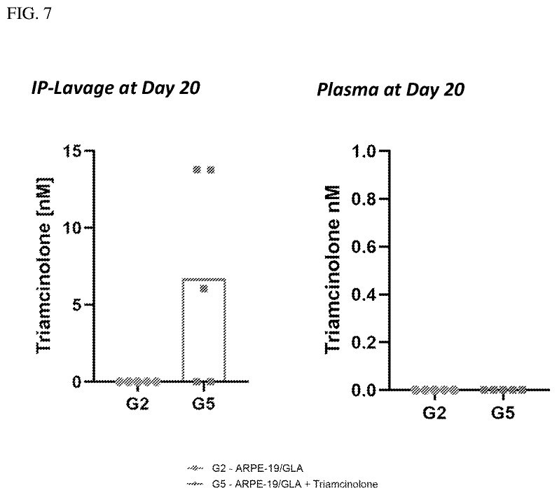

GLA-secreting human cells were co-encapsulated with TAH crystals into spheres that were then implanted in the peritoneum of immune-competent C57Bl/6 mice. The amount of hGLA produced by the spheres (e.g., devices) in vivo at site of implantation and in systemic circulation was measured. Significant levels of hGLA were found both in the peritoneal lavage and plasma 20 days post-implantation, as shown in FIG. 6. Further analyses of retrieved spheres demonstrated that the GLA-secreting human cells co-encapsulated with TAH maintained their viability in vivo over a course of 20 days (data not shown).

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. An implantable device comprising (a) at least one cell-containing compartment comprising a plurality of living cells and (b) an extended release formulation of a glucocorticoid compound, wherein the extended release formulation and device are configured to provide continuous, local release of the glucocorticoid compound from the device during a release period (e.g., at least 7 days) after implantation into an immune-competent subject.

2. The implantable device of claim 1, wherein the device comprises at least one of the following features:
(i) the glucocorticoid compound is released from the implanted device in an amount effective to inhibit PFO formation on the device during the entire release period;
(ii) an afibrotic compound disposed on the exterior surface of the device;
(iii) the exterior surface of the device does not comprise any alginate;
(iv) all of the cells in the plurality of living cells are mammalian cells genetically modified to express and secrete a first therapeutic substance;
(v) a second plurality of living mammalian cells genetically modified to express and secrete a second therapeutic substance that is different than the first substance;
(vi) the first therapeutic substance secreted by the living cells is a protein comprising a heterologous signal peptide sequence, optionally the signal peptide sequence consists essentially of MGWRAAGALLLALLLHGRLLA (SEQ ID NO:13);
(vii) the living cells are derived from ARPE-19 cells;
(viii) the living cells are derived from a non-human mammalian cell line; and
(ix) the living cells are derived from an induced pluripotent stem cell line.

3. The device of claim 1, wherein the release period is at least any of 10 days, 15 days, 30 days, 60 days, or 90 days.

4. The implantable device of claim 2, which comprises feature (i) or feature (ii).

5. The implantable device of claim 2, which comprises feature (i) and feature (ii) and optionally feature (iii).

6. The implantable device of claim 2, which comprises feature (vii) and optionally feature (vi).

7. The implantable device of claim 1, wherein the glucocorticoid compound is a compound of Formula (IV): 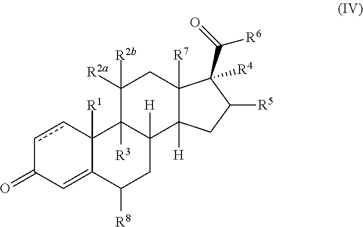
or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

8. The implantable device of claim 6, wherein the glucocorticoid compound is selected from the group consisting of triamcinolone hexacetonide, triamcinolone acetonide, fluticasone furoate, fluticasone propionate, mometasone furoate, and beclomethasone diproprionate.

9. The implantable device of claim 1, wherein the cell-containing compartment is surrounded by a barrier compartment.

10. The implantable device of claim 9, wherein the barrier compartment comprises a hydrogel-forming polymer, e.g., an alginate.

11. The implantable device of claim 2, wherein the afibrotic compound is a compound of Formula (I): 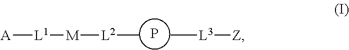
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{μl} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

12. The implantable device of claim 1, wherein the cell-containing compartment comprises a hydrogel-forming polymer, e.g., an alginate, e.g., a GRGDSP-modified alginate.

13. The implantable device of claim 11, wherein the compound of Formula (I) is: 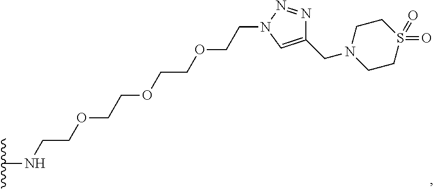 [SMILES: *NCCOCCOCCOCCn1cc(CN2CCS(=O)(=O)CC2)nn1]
or a pharmaceutically acceptable salt thereof.

14. The implantable device of claim 11, wherein the compound of Formula (I) is: 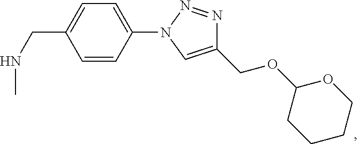 [SMILES: CNCc1ccc(-n2cc(COC3CCCCO3)nn2)cc1]
or a pharmaceutically acceptable salt thereof.

15. A hydrogel capsule comprising:
(a) a cell-containing compartment which comprises living cells encapsulated in a first polymer composition,
(b) a barrier compartment surrounding the cell-containing compartment and comprising a second polymer composition; and
(c) an extended release formulation of a glucocorticoid compound.

16. The hydrogel capsule of claim 15, wherein the extended release formulation is present in one or both of the cell-containing compartment and the barrier compartment.

17. The hydrogel capsule of claim 16, wherein the barrier compartment is substantially free of the extended release formulation.

18. The hydrogel capsule of claim 15, wherein the glucocorticoid compound is a compound of Formula (IV): 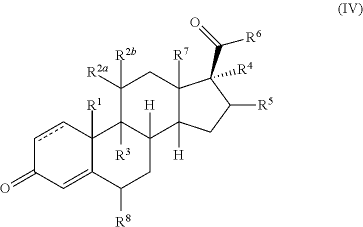
or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

19. The hydrogel capsule of claim 15, wherein the glucocorticoid is selected from the group consisting of triamcinolone hexacetonide, triamcinolone acetonide, fluticasone furoate, fluticasone propionate, mometasone furoate, and beclomethasone diproprionate.

20. The hydrogel capsule of claim 15, wherein the glucocorticoid compound is selected from the group consisting of:
(i) triamcinolone or an ester derivative thereof (e.g., triamcinolone hexacetonide (TAH), triamcinolone acetonide, triamcinolone benetonide, triamcinolone diacetate);
(ii) fluticasone or an ester derivative thereof (e.g., fluticasone furoate (FF), fluticasone propionate); and
(iii) mometasone or an ester derivative thereof (e.g., mometasone furoate (MF)).

21. The hydrogel capsule of claim 15, wherein the glucocorticoid compound is TAH or FF.

22. The hydrogel capsule of claim 15, wherein the extended release formulation comprises particles of a solid form of the glucocorticoid compound, wherein the solid form is an amorphous solid, a crystalline solid, or a mixture thereof.

23. The hydrogel capsule of claim 15, wherein the second polymer composition comprises an alginate covalently modified with at least one compound of Formula (I) (e.g., as described herein).

24. The hydrogel capsule of claim 23, wherein the compound is selected from the group consisting of Compound 100, Compound 101, Compound 110, Compound 112, Compound 113, Compound 114, Compound 122 and Compound 123 shown in Table 2 above, or a pharmaceutically acceptable salt of the compound.

25. The hydrogel capsule of claim 15, wherein the hydrogel capsule has a spherical shape and has a diameter of 0.5 millimeter to 5 millimeters, optionally a diameter of about 1 mm to about 2 mm, or about 1.3 mm to about 1.7 mm or about 1.5 mm.

26. The hydrogel capsule of claim 15, wherein the first polymer composition comprises a hydrogel-forming polymer (e.g., an alginate, a GRGDSP-modified alginate) and the extended release formulation of the glucocorticoid compound is prepared by a process which comprises adding a desired quantity of an amorphous powder of the glucocorticoid compound to a desired volume of a solution comprising the hydrogel-forming polymer, sonicating the resulting mixture until a substantially homogenous suspension is formed, adding the living cells to the suspension and contacting droplets of the polymer, glucocorticoid compound and cell suspension with a cross-linking solution.

27. The hydrogel capsule of claim 26, wherein the quantity of the amorphous powder and the volume of the polymer solution are selected to achieve a mixture of 2.5 mg to 5.0 mg powder per mL polymer solution.

28. The hydrogel capsule of claim 15, wherein the barrier compartment has an average thickness of about 10 to about 300 microns, about 20 to about 150 microns, or about 40 to about 75 microns.

29. The hydrogel capsule of claim 15, wherein the second polymer composition in the barrier compartment comprises a mixture of the modified alginate and an unmodified alginate.

30. The hydrogel capsule of claim 15, wherein at least a portion of the living cells are genetically modified to continuously express and secrete an IL-10 protein.

31. The hydrogel capsule of claim 15, wherein at least a portion of the living cells are genetically modified to continuously express and secrete a therapeutic protein or therapeutic peptide.

32. The hydrogel capsule of claim 15, wherein at least a portion of the living cells are genetically modified to express and secrete a therapeutic protein or therapeutic peptide, optionally wherein the therapeutic protein is a cytokine, an enzyme, a hormone, or a blood clotting factor.

33. The hydrogel capsule of claim 15, wherein all of the living cells in the capsule are derived from ARPE-19 cells.

34. A device composition comprising a preparation of hydrogel capsules and a pharmaceutically acceptable excipient, wherein each hydrogel capsule in the preparation is a hydrogel capsule as defined in claim 15.

35. The device composition of claim 34, which has a volume of less than 10 milliliters, optionally less than 8 ml, or less than 5 ml.

36. A method of treating a subject in need of therapy with a therapeutic substance, comprising administering to the subject the device of claim 1, the hydrogel capsule of claim 15, or the device composition of claim 34, wherein the living cells are genetically modified to express and secrete the therapeutic substance.

37. The method of claim 36, wherein the device, capsule or device composition is administered by implantation into the peritoneal cavity of the subject.

38. The method of claim 36, wherein the glucocorticoid compound is TAH, FF, or MF and each device in the device composition is a two-compartment hydrogel capsule of about 1 mm to 2 mm in diameter, and comprises an inner compartment comprising the genetically modified mammalian cells and the extended release formulation of TAH, FF or MF and an outer compartment comprising an alginate modified with: 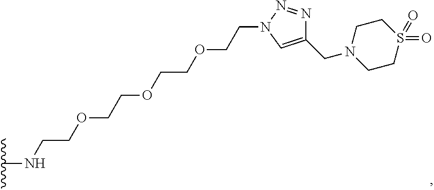 [SMILES: *NCCOCCOCCOCCn1cc(CN2CCS(=O)(=O)CC2)nn1]
or a pharmaceutically acceptable salt thereof.

39. The method of claim 35, wherein the genetically modified mammalian cells are xenogeneic to the subject.


SEQUENCE LISTING (22 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 537 bp, Artificial Sequence
  Description of Artificial Sequence Synthetic polynucleotide
atgcactctagcgccctgctgtgttgtctggtgctgctgacaggcgtcagagcctctcctggacagggaacccagagcgagaatagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactaa

SEQ ID NO: 3 - protein, 181 aa, Homo sapiens
MGWRAAGALLLALLLHGRLLASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - DNA, 546 bp, Artificial Sequence
  Description of Artificial Sequence Synthetic polynucleotide
atgggctggcgcgccgccggcgccctgctgctggccctgctgctgcacggccgcctgctggccagccccggccagggcacccagagcgagaacagctgcacccacttccccggcaacctgcccaacatgctgcgcgacctgcgcgacgccttcagccgcgtgaagaccttcttccagatgaaggaccagctggacaacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgcgcctgcgcctgcgccgctgccaccgcttcctgccctgcgagaacaagagcaaggccgtggagcaggtgaagaacgccttcaacaagctgcaggagaagggcatctacaaggccatgagcgagttcgacatcttcatcaactacatcgaggcctacatgaccatgaagatccgcaactaa

SEQ ID NO: 5 - DNA, 546 bp, Artificial Sequence
  Description of Artificial Sequence Synthetic polynucleotide
atgggctggcgagctgctggtgcacttctgctggctctgctgcttcatggcagactgctggcttctcctggacagggcacacagagcgagaacagctgcacacacttccccggcaacctgcctaacatgctgcgggatctgagagatgccttcagccgcgtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactaa

SEQ ID NO: 6 - protein, 178 aa, Artificial Sequence
  Description of Artificial Sequence Synthetic polypeptide
MHSSALLCCLVLLTGVRASPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENKSKAVEQVKNAFNKLQEKGIYKAMSEFDIFINYIEAYMTMKIRN

SEQ ID NO: 7 - DNA, 534 bp, Artificial Sequence
  Description of Artificial Sequence Synthetic polynucleotide
atgcactctagcgccctgctgtgttgtctggtgctgctgacaggcgtcagagcctctcctggacagggaacccagagcgagaatagctgcacccactttccaggcatcctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgatcagcctgggcgagaacctgaataccctgcggctgagactgcggagatgccacagactgctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaac

SEQ ID NO: 8 - protein, 18 aa, Artificial Sequence
  Description of Artificial Sequence Synthetic peptide
MKWVTFISLLFLFSSAYS

SEQ ID NO: 9 - protein, 20 aa, Artificial Sequence
  Description of Artificial Sequence Synthetic peptide
MVLQTQVFISLLLWISGAYG

SEQ ID NO: 10 - protein, 23 aa, Artificial Sequence
  Description of Artificial Sequence Synthetic peptide
MQMSPALTCLVLGLALVFGEGSA

SEQ ID NO: 11 - protein, 18 aa, Artificial Sequence
  Description of Artificial Sequence Synthetic peptide
MGLAWGLGVLFLMHVCGT

SEQ ID NO: 12 - protein, 31 aa, Artificial Sequence
  Description of Artificial Sequence Synthetic polypeptide
MLRGPGPGLLLLAVQCLGTAVPSTGASKSKR

SEQ ID NO: 13 - protein, 21 aa, Artificial Sequence
  Description of Artificial Sequence Synthetic peptide
MGWRAAGALLLALLLHGRLLA

SEQ ID NO: 14 - protein, 29 aa, Artificial Sequence
  Description of Artificial Sequence Synthetic peptide
MAGRSHPGPLRPLLPLLVVAACVLPGAGG

SEQ ID NO: 15 - protein, 19 aa, Artificial Sequence
  Description of Artificial Sequence Synthetic peptide
MEFGLSWVFLVALFRGVQC

SEQ ID NO: 16 - protein, 22 aa, Artificial Sequence
  Description of Artificial Sequence Synthetic peptide
MKYLLPTAAAGLLLLAAQPAMA

SEQ ID NO: 17 - protein, 23 aa, Artificial Sequence
  Description of Artificial Sequence Synthetic peptide
MRPTWAWWLFLVLLLALWAPARG

SEQ ID NO: 18 - protein, 21 aa, Artificial Sequence
  Description of Artificial Sequence Synthetic peptide
MWWRLWWLLLLLLLLWPMVWA

SEQ ID NO: 19 - protein, 17 aa, Artificial Sequence
  Description of Artificial Sequence Synthetic peptide
MGVKVLFALICIAVAEA

SEQ ID NO: 20 - protein, 24 aa, Artificial Sequence
  Description of Artificial Sequence Synthetic peptide
MPSSVSWGILLLAGLCCLVPVSLA

SEQ ID NO: 21 - protein, 18 aa, Artificial Sequence
  Description of Artificial Sequence Synthetic peptide
MHSSALLCCLVLLTGVRA

SEQ ID NO: 22 - protein, 6 aa, Artificial Sequence
  Description of Artificial Sequence Synthetic peptide
GRGDSP